(19) United States
(12) Patent Application Publication
(10) Pub. No.: US 2025/0215109 A1
(43) Pub. Date: Jul. 3, 2025

(54) ANTI-HER2/ANTI-CD47 MOLECULES AND USES THEREOF

(71) Applicant: Wuxi Biologics Ireland Limited, Dundalk (IE)

(72) Inventors: Siwei NIE, Shanghai (CN); Jiaxiang SHAO, Shanghai (CN); Qin MEI, Shanghai (CN); Fagen HU, Shanghai (CN); Jianqing XU, Shanghai (CN); Shuang WANG, Shanghai (CN); Jijie GU, Shanghai (CN); Yuhong SHEN, Shanghai (CN); Zhijian CHEN, Shanghai (CN); John Fenyu JIN, Shanghai (CN); Haopeng RUI, Shanghai (CN)

(21) Appl. No.: 18/850,531

(22) PCT Filed: Mar. 16, 2023
(86) PCT No.: PCT/CN2023/081820

§ 371 Date: Sep. 24, 2024

(30) Foreign Application Priority Data
Mar. 25, 2022 (WO) PCT/CN2022/082951

(51) Int. Cl. C07K 16/32; A61K 39/00; A61P 35/00; C07K 16/28

(52) U.S. Cl. CPC C07K 16/32; A61P 35/00; C07K 16/2803; A61K 2039/505; C07K 2317/24; C07K 2317/31; C07K 2317/51; C07K 2317/515; C07K 2317/522; C07K 2317/53; C07K 2317/565; C07K 2317/73; C07K 2317/732; C07K 2317/734; C07K 2317/92; C07K 2317/94

(57) ABSTRACT

Provided herein are anti-CD47/anti-HER2 polypeptide complexes, nucleic acid molecules encoding the variable domains of the polypeptide complexes, expression vectors and host cells used for the expression of the polypeptide complexes. The disclosure further provides methods for producing the polypeptide complexes and uses thereof.

### CROSS-REFERENCE

This application claims priority to International Patent Application No. PCT/CN2022/082951, filed on Mar. 25, 2022. The entire contents of the application are incorporated herein by reference.

### FIELD

This application generally relates to antibodies. More specifically, the application relates to bispecific antibodies that specifically bind to HER2 and CD47, methods for preparing the same, and uses thereof.

### BACKGROUND

Cluster of differentiation 47 (CD47) is a ˜50 kDa immunoglobulin superfamily membrane protein consisting of a single extracellular V-set IgSF domain, a presenilin domain with five membrane-spanning sections and a short cytoplasmic domain. CD47 interacts with its ligand, signal regulatory protein alpha (SIRPα) expressed on myeloid cells such as macrophages, and then acting an anti-phagocytic (“don't eat me”) signal to evade immune surveillance [1-2]. CD47 is a ubiquitous cell surface glycoprotein expressed on most normal cell types and is overexpressed on various malignant tumors including acute myeloid leukemia (AML), Non-Hodgkin's lymphoma (NHL), non-small cell lung cancer (NSCC), breast cancer (BC) and gastric cancer (GC). Thus, CD47 might serve as an innate immune checkpoint target for cancer therapy by blocking the CD47-SIRPα interaction to switch off the “don't eat me” signal [3].

HER2 (also known as erb-b2 receptor tyrosine kinase 2, or ERBB2) is a member of the epidermal growth factor receptor (EGFR) family, along with HER1 (also known as EGFR), HER3 and HER4. These receptors, functioning as homo- or heterodimers, activate multiple cellular pathways such as the mitogen-activated protein kinase (MAPK) and the phosphatidylinositol-3-kinase (PI3K) pathways, and then stimulate cell growth, survival and differentiation [4].

High CD47 expression is associated with poor prognosis in patients with various cancers, and co-expression of CD47 and Her2 may contribute to the disease progression in Her2⁺ cancers (e.g., BC, GC) post Her2-targeted therapy [5-6]. A specially designed CD47×Her2 bispecific antibody (BsAb) might preferentially target Her2+/CD47+ double positive tumor cells and minimize the effect on CD47 single positive normal cells to reduce systemic CD47 antigen mediated sink effect and hematological toxicity. Blockade of CD47/SIRPα signaling could enhance the anti-tumor efficacy of Her2-targeted therapy by increasing antibody-dependent cellular phagocytosis (ADCP) of tumor cells and further stimulate adaptive immunity. Further, the IgG1 Fc of BsAb could maintain antibody-dependent cellular cytotoxicity (ADCC) on Her2⁺ tumor cells.

Although Her2 over-expressing metastatic breast and gastric cancer patients initially respond to the Her2-targeted treatment, the majority of advanced-stage Her2-positive solid tumor (eg, breast cancer) patients who initially respond to trastuzumab eventually acquire resistance to treatment and relapse, despite persistence of Her2 gene amplification or overexpression, and there is a high unmet medical need for Her2-positive relapsed/refractory cancer patients. Therefore, CD47×Her2 BsAb might provide a novel treatment option in Her2-positive BC, GC and other solid tumors.

The present disclosure provides bispecific antibodies dual-targeting CD47 and HER2 and blocking CD47 and HER2 dual functions.

### SUMMARY

These and other objectives are provided for by the present disclosure which, in a broad sense, is directed to compounds, methods, compositions and articles of manufacture that provide antibodies with improved efficacy. The benefits provided by the present disclosure are broadly applicable in the field of antibody therapeutics and diagnostics and may be used in conjunction with other therapeutic and diagnostic agents, such as antibodies that react with a variety of targets.

The present disclosure provides bispecific polypeptide complexes or bispecific antibodies against CD47 and HER2. It also provides the nucleic acid molecules encoding the anti-CD47/anti-HER2 antibodies, expression vectors and host cells used for the expression of bispecific antibodies. The present disclosure further provides methods for preparing anti-CD47/anti-HER2 antibodies (CD47×Her2 BsAb), and for validating their functions in vivo and in vitro. The bispecific antibodies of the present disclosure provide a very potent agent for preventing or treating diseases comprising proliferative disorders and immune disorders.

In one aspect, the present disclosure provides a bispecific polypeptide complex or antigen-binding portion thereof, comprising a first antigen-binding moiety that specifically binds to HER2 (i.e., HER2-binding moiety) and a second antigen-binding moiety that specifically binds to CD47 (i.e., CD47-binding moiety).

In some embodiments, the present disclosure provides a bispecific polypeptide complex or antigen-binding portion thereof, comprising a first antigen-binding moiety that specifically binds to HER2 (i.e., HER2-binding moiety) and a second antigen-binding moiety that specifically binds to CD47 (i.e., CD47-binding moiety), wherein the first antigen-binding moiety comprises:
a heavy chain complementarity determining region (HCDR) 1 comprising the amino acid sequence of SEQ ID NO: 1, a HCDR2 comprising the amino acid sequence of SEQ ID NO: 2, a HCDR3 comprising the amino acid sequence of SEQ ID NO: 3, a light chain complementarity determining region (LCDR) 1 comprising the amino acid sequence of SEQ ID NO: 4, a LCDR2 comprising the amino acid sequence of SEQ ID NO: 5, and a LCDR3 comprising the amino acid sequence of SEQ ID NO: 6; and
the second antigen-binding moiety comprises:
a HCDR1 comprising the amino acid sequence of SEQ ID NO: 7, a HCDR2 comprising the amino acid sequence of SEQ ID NO: 8, a HCDR3 comprising the amino acid sequence of SEQ ID NO: 9, a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

In some embodiments, the first antigen-binding moiety and the second antigen-binding moiety are in Fab format.

In some embodiments, the first antigen-binding moiety comprises a first heavy chain variable domain (VH1) operably linked to a first T cell receptor (TCR) constant region (referred to as C1 or CBeta), and a first light chain variable domain (VL1) operably linked to a second TCR constant region (referred to as C2 or CAlpha); and the second antigen-binding moiety comprises a second VH (VH2) operably linked to an antibody heavy chain CH1 domain and a second VL (VL2) operably linked to an antibody light chain constant (CL) domain, wherein C1 and C2 are capable of forming one or more non-native interchain disulfide bond(s). The positions of C1 and C2 domains in the bispecific polypeptide complex or antigen-binding portion thereof may be exchanged.

In some other embodiments, the first antigen-binding moiety comprises a first heavy chain variable domain (VH1) operably linked to an antibody heavy chain CH1 domain and a first light chain variable domain (VL1) operably linked to an antibody light chain constant (CL) domain; and the second antigen-binding moiety comprises a second VH (VH2) operably linked to a first T cell receptor (TCR) constant region (C1), and a second VL (VL2) operably linked to a second TCR constant region (C2), wherein C1 and C2 are capable of forming one or more non-native interchain disulfide bond(s). The positions of C1 and C2 domains in the bispecific polypeptide complex or antigen-binding portion thereof may be exchanged.

In some embodiments, the first VH comprises the amino acid sequence of SEQ ID NO: 13 or an amino acid sequence with at least 85%, 90%, or 95% identity to SEQ ID NO: 13 and the first VL comprises the amino acid sequence of SEQ ID NO:14 or an amino acid sequence with at least 85%, 90%, or 95% identity to SEQ ID NO: 14; and/or
the second VH comprises the amino acid sequence of SEQ ID NO: 15 or an amino acid sequence with at least 85%, 90%, or 95% identity to SEQ ID NO: 15 and the second VL comprises the amino acid sequence of SEQ ID NO:16 or an amino acid sequence with at least 85%, 90%, or 95% identity to SEQ ID NO: 16.

The bispecific polypeptide complex may further comprise a Fc region, such as a human IgG (IgG1, IgG2, IgG3 or IgG4) Fc region, such as a human IgG1 Fc region, IgG2 Fc region or IgG4 Fc region. The Fc region may be a native Fc region or an engineered Fc region. For example, the human IgG1 Fc region may be engineered to comprise a “knob into hole” structure or other modifications conventionally known in the art. Optionally, the Fc region may be selected from one of the following: (a) a human IgG1 Fc region engineered to comprise a “knob into hole” structure; (b) a human IgG4 Fc region engineered to comprise a “knob into hole” structure and a S228P mutation; and (c) a human IgG4 Fc region engineered to comprise a S228P mutation and M252Y/S254T/T256E mutations.

In some embodiments, the bispecific polypeptide complex comprises one CD47-binding moiety and one HER2-binding moiety. For example, the bispecific polypeptide complex comprises two heavy chains and two light chains, wherein the first heavy chain comprises domains operably linked as in VH1-C1-hinge-Fc from the N terminus to the C terminus, the second heavy chain comprises domains operably linked as in VH2-CH1-hinge-Fc from the N terminus to the C terminus, the first light chain comprises domains operably linked as in VL1-C2 from the N terminus to the C terminus, and the second light chain comprises domains operably linked as in VL2-CL from the N terminus to the C terminus. As an alternative example of the bispecific polypeptide complex, from the N terminus to the C terminus, the first heavy chain comprises domains operably linked as in VH1-CH1-hinge-Fc, the second heavy chain comprises domains operably linked as in VH2-C1-hinge-Fc, the first light chain comprises domains operably linked as in VL1-CL, and the second light chain comprises domains operably linked as in VL2-C2. The positions of C1 and C2 domains in the bispecific polypeptide complex or antigen-binding portion thereof may be exchanged.

In some embodiments, the bispecific polypeptide complex as disclosed herein comprises:
a first and a second heavy chain that comprise SEQ ID NOs: 19 and 20, respectively, a first and a second light chain that comprise SEQ ID NOs: 21 and 22, respectively.

In some embodiments, the bispecific polypeptide complex comprises two CD47-binding moieties and two HER2-binding moieties. For example, the bispecific polypeptide complex comprises two heavy chains and four light chains, wherein from the N-terminus to the C-terminus: the first and second heavy chains each comprises domains operably linked as in VH1-C1-VH2-CH1-hinge-Fe, VH2-CH1-VH1-C1-hinge-Fe, VH1-C1-hinge-Fc-VH2-CH1, or VH2-CH1-hinge-Fc-VH1-C1, the first and second light chains each comprises domains operably linked as in VL1-C2, and the third and fourth light chains each comprises domains operably linked as in VL2-CL. Alternatively, the bispecific polypeptide complex comprises two heavy chains and four light chains, wherein from the N-terminus to the C-terminus: the first and second heavy chains each comprises domains operably linked as in VH1-CH1-VH2-C1-hinge-Fc, VH2-C1-VH1-CH1-hinge-Fc, VH1-CH1-hinge-Fc-VH2-C1, or VH2-C1-hinge-Fc-VH1-CH1, the first and second light chains each comprises domains operably linked as in VL1-CL, and the third and fourth light chains each comprises domains operably linked as in VL2-C2. The positions of C1 and C2 domains in the bispecific polypeptide complex or antigen-binding portion thereof may be exchanged.

In some embodiments, the bispecific polypeptide complex comprises one CD47-binding moiety and two HER2-binding moieties. For example, the bispecific polypeptide complex comprises two heavy chains and three light chains, wherein from the N-terminus to the C-terminus: the first heavy chain comprises domains operably linked as in VH2-CH1-VH1-C1-hinge-Fc or VH1-C1-hinge-Fc-VH2-CH1, the second heavy chain comprises domains operably linked as in VH1-C1-hinge-Fc, the first and second light chains each comprises domains operably linked as in VL1-C2, and the third light chain comprises domains operably linked as in VL2-CL. Alternatively, the first heavy chain comprises domains operably linked as in VH2-C1-VH1-CH1-hinge-Fc or VH1-CH1-hinge-Fc-VH2-C1, the second heavy chain comprises domains operably linked as in VH1-CH1-hinge-Fc, the first and second light chains each comprises domains operably linked as in VL1-CL, and the third light chain comprises domains operably linked as in VL2-C2. The positions of C1 and C2 domains in the bispecific polypeptide complex or antigen-binding portion thereof may be exchanged.

In some embodiments, the bispecific polypeptide complex comprises two CD47-binding moieties and one HER2-binding moiety. For example, the bispecific polypeptide complex comprises two heavy chains and three light chains, wherein from the N-terminus to the C-terminus: the first heavy chain comprises domains operably linked as in VH1-C1-VH2-CH1-hinge-Fc or VH2-CH1-hinge-Fc-VH1-C1, the second heavy chain comprises domains operably linked as in VH2-CH1-hinge-Fe, the first light chain comprises domains operably linked as in VL1-C2, and the second and third light chains each comprises domains operably linked as in VL2-CL. Alternatively, the first heavy chain comprises domains operably linked as in VH1-CH1-VH2-C1-hinge-Fc or VH2-C1-hinge-Fc-VH1-CH1, the second heavy chain comprises domains operably linked as in VH2-C1-hinge-Fe, the first light chain comprises domains operably linked as in VL1-CL, and the second and third light chains each comprises domains operably linked as in VL2-C2. The positions of C1 and C2 domains in the bispecific polypeptide complex or antigen-binding portion thereof may be exchanged.

The operably linkage or “-” may be via a direct linkage or via a peptide linker, such as a GS linker, for example (GS)n, (GGS)n, (GGGS)n, (GGGGS)n, (GGSG)n, (GGGSS)n, wherein n is an integer of 1-9. The bispecific polypeptide complex may be a humanized antibody or fully human antibody. In some embodiments, the bispecific polypeptide complex is a fully human antibody.

In one aspect, disclosed herein is an isolated nucleic acid molecule, comprising a nucleic acid sequence encoding the bispecific polypeptide complex or antigen-binding portion thereof.

In one aspect, disclosed herein is a vector comprising the nucleic acid molecule as described above. In one aspect, disclosed herein is a host cell comprising the nucleic acid molecule or vector as described above.

In one aspect, disclosed herein is a pharmaceutical composition comprising the bispecific polypeptide complex or antigen-binding portion thereof and a pharmaceutically acceptable carrier.

In one aspect, disclosed herein is a method for producing the bispecific polypeptide complex, comprising the steps of:
culturing a host cell comprising the nucleic acid sequence encoding the polypeptide complex under suitable conditions; and
isolating the polypeptide complex from the host cell.

In one aspect, disclosed herein is a method for modulating a HER2 and/or CD47 related immune response in a subject, comprising administering to the subject the bispecific polypeptide complex or antigen-binding portion thereof or the pharmaceutical composition as disclosed herein to the subject.

In one aspect, disclosed herein is a method for inhibiting growth of tumor cells, such as tumor cells that are CD47 and/or HER2 positive, in a subject, comprising administering an effective amount of the bispecific polypeptide complex or antigen-binding portion thereof or the pharmaceutical composition as disclosed herein to the subject.

In one aspect, disclosed herein is a method for inducing macrophage-mediated phagocytosis of tumor cells in a subject, comprising administering an effective amount of the bispecific polypeptide complex or antigen-binding portion thereof or the pharmaceutical composition as disclosed herein to the subject.

In one aspect, disclosed herein is a method for inducing natural killer cell-mediated cytotoxicity toward (or against) tumor cells in a subject, comprising administering an effective amount of the bispecific polypeptide complex or antigen-binding portion thereof or the pharmaceutical composition as disclosed herein to the subject.

In one aspect, disclosed herein is a method for diagnosing, preventing or treating cancer in a subject, comprising administering an effective amount of the bispecific polypeptide complex or antigen-binding portion thereof or the pharmaceutical composition to the subject. In some embodiments, the cancer is a HER2 and/or CD47 positive cancer and selected from colon cancer, colorectal cancer, breast cancer, lung cancer including NSCLC and small cell lung carcinoma, cervical cancer, renal cancer, glioblastoma, ovarian cancer, pancreatic cancer, prostate cancer, esophageal cancer, gastric cancer, melanoma, liver cancer, head and neck cancer, skin cancer, bladder cancer, brain cancer, bronchial cancer, cancer of the bile duct, endometrial cancer, ependymoma, glioma, cancer of unknown primary origin, medulloblastoma, nasopharygeal cancer, neuroblastoma, squamous cell carcinoma, retinoblastoma, sarcoma, testicular cancer, thymoma, thyroid cancer, urachal cancer, uterine cancer, vaginal cancer, astrocytoma, basal cell carcinoma, and combinations thereof.

In some further embodiments, the cancer is breast cancer, gastric cancer, lung cancer, skin cancer, or colorectal cancer.

In one aspect, disclosed herein is a composition for use in diagnosing, preventing or treating cancer in a subject, comprising an effective amount of the bispecific polypeptide complex or antigen-binding portion thereof disclosed herein.

In one aspect, disclosed herein is a bispecific polypeptide complex or antigen-binding portion thereof for use
i) in the modulation of a HER2 and/or CD47 related immune response; and/or
ii) in inducing macrophage-mediated phagocytosis of tumor cells; and/or
iii) in inducing natural killer cell-mediated cytotoxicity of tumor cells; and/or
iv) in inhibiting growth of tumor cells, such as tumor cells that are CD47 and/or HER2 positive.

In one aspect, disclosed herein is the bispecific polypeptide complex or antigen-binding portion thereof for use in diagnosing, treating or preventing cancer.

In one aspect, disclosed herein is use of the bispecific polypeptide complex or antigen-binding portion thereof in the manufacture of a medicament for
i) modulating a HER2 and/or CD47 related immune response;
ii) inducing macrophage-mediated phagocytosis of tumor cells;
iii) inducing natural killer cell-mediated cytotoxicity of tumor cells; and/or
iv) inhibiting growth of tumor cells, such as tumor cells that are CD47 and/or HER2 positive.

In one aspect, disclosed herein is use of the bispecific polypeptide complex or antigen-binding portion thereof in the manufacture of a medicament for diagnosing, treating or preventing cancer.

The bispecific polypeptide complex or antigen-binding portion thereof as disclosed herein may be administered in combination with a chemotherapeutic agent, radiation and/or other agents for use in cancer immunotherapy, and may be for use in a combinatory therapy together with a chemotherapeutic agent, radiation and/or other agents for use in cancer immunotherapy.

In one aspect, disclosed herein is a kit, wherein the kit comprises a container comprising the bispecific polypeptide complex or antigen-binding portion thereof as described above.

The foregoing is a summary and thus contains, by necessity, simplifications, generalizations, and omissions of detail; consequently, those skilled in the art will appreciate that the summary is illustrative only and is not intended to be in any way limiting.

### BRIEF DESCRIPTION OF THE FIGURES

FIG. 1 illustrates a schematic representation of W308032 bispecific antibody in WuXiBody E17 format. From N terminus to C terminus, 1^{st} heavy chain, VH1-Cbeta-hinge-Fc; 2^{nd} heavy chain, VH2-CH1-hinge-Fc; 1^{st} light chain, VL1-CAlpha; and 2^{nd} light chain, VL2-CL.

FIGS. 2-3 illustrate SDS-PAGE and SEC-HPLC characterizations of W308032-U5T6.E17-57.uIgG1, respectively.

FIGS. 4-6 show the DSF, DLS and HIC-HPLC characterizations of W308032-U5T6.E17-57.uIgG1, respectively.

FIGS. 7A-7F show the binding affinity of W308032-U5T6.E17-57.uIgG1 on human Her2 protein (A: round #1, B: round #2, C: round #3) and on human CD47 protein (D: round #1, E: round #2, F: round #3), respectively, determined by SPR.

FIGS. 8A-8B show binding of W308032-U5T6.E17-57.uIgG1 on SK-BR-3 without antigen sink (FIG. 8A), or after being sunk by human blood cells (FIG. 8B).

FIGS. 9A-9B show binding of W308032-U5T6.E17-57.uIgG1 on SK-BR-3 without antigen sink (FIG. 9A), or after being sunk by Jurkat cells (FIG. 9B).

FIGS. 10A-10B show binding of W308032-U5T6.E17-57.uIgG1 on HCC1954 without antigen sink (FIG. 10A), or after being sunk by Jurkat cells (FIG. 10B).

FIG. 11 shows binding of W308032-U5T6.E17-57.uIgG1 on human blood cells.

FIG. 12 shows binding of W308032-U5T6.E17-57.uIgG1 on Jurkat cells.

FIGS. 13A-13B show CD47 ligand blocking of W308032-U5T6.E17-57.uIgG1 on SK-BR-3 without antigen sink (FIG. 13A), or after being sunk by human blood cells (FIG. 13B).

FIGS. 14A-14B show CD47 ligand blocking of W308032-U5T6.E17-57.uIgG1 on SK-BR-3 without antigen sink (FIG. 14A), or after being sunk by Jurkat cells (FIG. 14B).

FIG. 15 shows CD47 ligand blocking of W308032-U5T6.E17-57.uIgG1 on Jurkat cells.

FIGS. 16A-16B show HER2 and CD47 occupancy of W308032-U5T6.E17-57.uIgG1 on SK-BR-3, respectively.

FIGS. 17A-17B show ADCP of W308032-U5T6.E17-57.uIgG1 on SK-BR-3 in two independent experiments.

FIGS. 18-19 show ADCP of W308032-U5T6.E17-57.uIgG1 on HCC1954 (FIG. 18) and NCI-N87 (FIG. 19), respectively.

FIGS. 20-21 show ADCP of W308032-U5T6.E17-57.uIgG1 on human blood cells (FIG. 20) and Jurkat cells (FIG. 21), respectively.

FIGS. 22-24 show ADCC of W308032-U5T6.E17-57.uIgG1 on SK-BR-3 (FIG. 22), HCC1954 (FIG. 23) and NCI-N87 (FIG. 24), respectively.

FIG. 25 shows ADCC of W308032-U5T6.E17-57.uIgG1 on Jurkat cells.

FIGS. 26-27 show inhibitory effect of W308032-U5T6.E17-57.uIgG1 on proliferation of SK-BR-3 (FIG. 26) and NCI-N87 (FIG. 27), respectively.

FIG. 28 shows hemagglutination of W308032-U5T6.E17-57.uIgG1 on human blood cells.

FIG. 29 shows the thermal stability results for W308032-U5T6.E17-57.uIgG1.

FIG. 30 shows the combinatory anti-tumor effect of anti-HER2 and anti-CD47 therapies in a HCC1954 xenograft model.

FIG. 31 shows the anti-tumor effect of W308032-U5T6.E17-57.uIgG1 in a HCC1954 xenograft model at different dosage levels.

FIG. 32 shows the anti-tumor effect of a combo therapy with pertuzumab in a HCC1954 xenograft model.

FIG. 33 shows a summary of tumor weight on Day 36 in a HCC1954 xenograft model. Note: Two way ANOVA Bonferroni posttests, *p<0.05; **p<0.01.

FIG. 34 shows the combinatory anti-tumor effect of anti-Her2 and anti-CD47 therapies in NCI-N87 xenograft model.

FIG. 35 shows a summary of the anti-tumor effect of W308032-U5T6.E17-57.uIgG1 in NCI-N87 xenograft model.

FIG. 36 shows a summary of the anti-tumor effect of combo therapy with paclitaxel in NCI-N87 xenograft model.

FIG. 37 shows a summary of the anti-tumor effect of combo therapy with paclitaxel in JIMT-1 xenograft model.

FIG. 38 shows a summary of tumor rebound after last dosing. The dashed lines represent the mean initial tumor volume when grouping.

FIG. 39 shows a summary of rodent pharmacokinetics for W308032-U5T6.E17-57.uIgG1 and Trastuzumab.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a”, “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised”, is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are well known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are well known and commonly used in the art. Other aspects, features, and advantages of the methods, compositions and/or devices and/or other subject matter described herein will become apparent in the teachings set forth herein. Further, the contents of all references, patents and published patent applications cited throughout this application are incorporated herein in entirety by reference.

### Definitions

In order to better understand the disclosure, the definitions and explanations of the relevant terms are provided as follows.

The term “antibody” or “Ab”, as used herein, is used in the broadest sense, and encompasses any form of antibody that exhibits the desired biological or binding activity. It covers, but is not limited to, humanized antibodies, fully human antibodies, chimeric antibodies and single-domain antibodies. The bispecific polypeptide complexes as disclosed herein also belong to antibodies. A common antibody generally comprises heavy chain(s) and light chain(s). Heavy chains may be classified into μ, δ, γ, α and ε, which define isotypes of an antibody as IgM, IgD, IgG, IgA and IgE, respectively. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{L}). As demonstrated herein, various modifications may be made to the constant regions or they may be replaced with other immunoglobulin-derived constant regions. V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from the N-terminus to the C-terminus. The variable region (V_{H} and V_{L}) of each heavy/light chain pair forms antigen binding sites, respectively. The extent of the framework region and CDRs can be precisely identified using methodology known in the art, for example, by the Kabat definition, the Chothia definition, the AbM definition, the EU definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May, 63(1):78-85; and Almagro, J. Mol. Recognit. 17:132-143 (2004). See also hgmp.mrc.ac.uk and bioinforg.uk/abs. Correspondence or alignments between numberings according to different definitions can for example be found at www.imgt.org/ (see also Giudicelli V et al. IMGT, the international ImMunoGeneTics database. Nucleic Acids Res. (1997) 25:206-11; and Lefranc M P et al., IMGT unique numbering for immunoglobulin and T cell receptor variable domains and Ig superfamily V-like domains. Dev Comp Immunol. (2003) 27:55-77). Antibodies may be of different antibody isotypes, for example, IgG (e.g., IgG1, IgG2, IgG3 or IgG4 subtype), IgA1, IgA2, IgD, IgE or IgM antibody.

The term “antigen-binding moiety” as used herein refers to an antibody fragment formed from a portion of an antibody comprising one or more CDRs, or any other antibody fragment that binds to an antigen but does not comprise an intact native antibody structure. Examples of antigen-binding moiety include, without limitation, a variable domain, a variable region, a diabody, a Fab, a Fab′, a F(ab′)2, an Fv fragment, a single chain Fv fragment (scFv), a disulfide stabilized Fv fragment (dsFv), a (dsFv)2, a bispecific dsFv (dsFv-dsFv′), a disulfide stabilized diabody (ds diabody), a multispecific antibody, a camelized single domain antibody, a single variable domain (i.e. VHH), a nanobody, a domain antibody, and a bivalent domain antibody. An antigen-binding moiety is capable of binding to the same antigen to which the parent antibody binds. In certain embodiments, an antigen-binding moiety may comprise one or more CDRs from a particular human antibody grafted to a framework region from one or more different human antibodies. For more and detailed formats of antigen-binding moiety are described in Spiess et al, (2015) Molecular Immunology 67: 95-106, and Brinkman et al., mAbs, 9(2), pp.182-212 (2017), which are incorporated herein by their entirety.

The term “antigen-binding portion” or “antigen-binding fragment” of an antibody, which can be interchangeably used in the context of the application, refers to polypeptides comprising fragments of a full-length antibody or the polypeptide complex as disclosed herein, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes.

“Fab” with regard to an antibody refers to that portion of the antibody consisting of a single light chain (both variable and constant regions) associating to the variable region and first constant region of a single heavy chain by a disulfide bond. In certain embodiments, the constant regions of both the light chain and heavy chain are replaced with TCR constant regions.

“Fc” (short for fragment, crystallizable) with regard to an antibody refers to that portion of the antibody comprising the second (CH2) and third (CH3) constant regions of a first heavy chain bound to the second and third constant regions of a second heavy chain via disulfide bonding. The Fc region as used herein may also comprise part or whole of the hinge region. The term “hinge” or “hinge region” refers to a flexible amino acid stretch in the central part of the heavy chains of the IgG and IgA immunoglobulin classes, which links these 2 chains by disulfide bonds, and as used herein may include native hinge region as a whole, in partial, homologues or functional equivalents thereof. The Fc region of the antibody is responsible for various effector functions such as ADCC and CDC, but generally does not function in antigen binding. The capacity of antibodies to initiate and regulate effector functions through their Fc domain is a key component of their in vivo protective activity. Although the neutralizing activity of antibodies has been previously considered to be solely the outcome of Fab-antigen interactions, it has become apparent that their in vivo activity is highly dependent on interactions of the IgG Fc domain with its cognate receptors, Fey receptors (FcγRs), expressed on the surface of effector leukocytes.

The term “humanized antibody”, as used herein, refers to antibodies in which CDR sequences derived from the germline of another mammalian species, such as a mouse, have been grafted onto human framework sequences. Additional framework region modifications may be made within the human framework sequences.

The term “human antibody” or “fully human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The terms “operably link” and “operably linked” refer to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide. The term operably linked, when used herein to describe domains linked to form a polypeptide, can be represented by a “-”, and can refer to a direct linkage between domains or linkage via a linker comprising 1-30 amino acids in length, such as a single amino acid or a series of (G4S)n linker, with n=1-5 (1, 2, 3, 4, or 5).

The term “Ka”, as used herein, is intended to refer to the association rate of a particular antibody-antigen interaction, whereas the term “Kd” as used herein, is intended to refer to the dissociation rate of a particular antibody-antigen interaction. The term “KD” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the KD of an antibody is by using surface plasmon resonance, preferably using a biosensor system such as a Biacore® system.

The term “specific binding” or “specifically binds” as used herein refers to a non-random binding reaction between two molecules, such as for example between an antibody and an antigen. The term “high affinity” for an IgG antibody, as used herein, refers to an antibody having a KD of 1×10⁻⁷ M or less, more preferably 5×10⁻⁸ M or less, even more preferably 1×10⁻⁸ M or less, even more preferably 5×10⁻⁹ M or less, even more preferably 1×10⁻⁹ M or less, and even more preferably 5×10⁻¹⁰ M or less, for a target antigen, e.g., as measured by SPR.

The term “EC₅₀”, as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “IC₅₀”, as used herein, which is also termed as “half maximal inhibitory concentration” refers to the half maximal inhibitory concentration of a drug, antibody or other substances. It is a measure of the effectiveness of the drug, antibody or other substances in inhibiting biological or biochemical function. In the context of the application, IC₅₀ is expressed in the unit of “nM”.

The ability to “inhibit binding” or “block binding”, as used herein, refers to the ability of an antibody to inhibit the binding interaction between two molecules (e.g., human CD47 and CD47 ligand SIRPα) to any detectable degree. In some embodiments, the antibody as disclosed herein blocks binding between CD47 and SIRPPα with an IC₅₀ of no more than 1 nM, no more than 0.8 nM, no more than 0.6 nM, no more than 0.4 nM, or no more than 0.3 nM.

The term “epitope”, as used herein, refers to a portion on antigen that an immunoglobulin or antibody specifically binds to. “Epitope” is also known as “antigenic determinant”. Epitope or antigenic determinant generally consists of chemically active surface groups of a molecule such as amino acids, carbohydrates or sugar side chains, and generally has a specific three-dimensional structure and a specific charge characteristic. For example, an epitope generally comprises at least 3, 4, 5, 6, 7, 8, 9, 10, 11, 12, 13, 14 or 15 consecutive or non-consecutive amino acids in a unique steric conformation, which may be “linear” or “conformational”. See, for example, Epitope Mapping Protocols in Methods in Molecular Biology, Vol. 66, G. E. Morris, Ed. (1996).

The term “isolated”, as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody”, as used herein, is intended to refer to an antibody that is substantially free of other antibodies having different antigenic specificities (e.g., an isolated bispecific antibody that specifically binds CD47 and HER2 protein is substantially free of antibodies that have different targeted antigens or epitopes). An isolated antibody that specifically binds a human CD47 protein and HER2 protein may, however, have cross-reactivity to other antigens, such as CD47 or HER2 proteins from other species. Moreover, an isolated antibody can be substantially free of other cellular material and/or chemicals.

The term “vector”, as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as k phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell”, as used herein, refers to a cell into which a vector can be introduced, including, but not limited to, prokaryotic cell such as E. coli or Bacillus subtilis, fungal cell such as yeast cell or Aspergillus, insect cell such as S2 Drosophila cell or Sf9, and animal cell such as fibroblast, CHO cell, COS cell, NSO cell, HeLa cell, BHK cell, HEK 293 cell or human cell.

The term “identity”, as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity”, as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection” or “transfect”, as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197.

The term “SPR” or “surface plasmon resonance”, as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 and Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS”, as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017 Nov. 9.). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” includes any human or nonhuman animal, preferably humans.

The term “associated with HER2” or “related to HER2”, as used herein with respect to a disease or condition, refers to any disease or condition that is caused by, exacerbated by, or otherwise linked to increased or decreased expression or activities of HER2 (e.g., a human HER2).

The term “cancer”, as used herein, refers to any or a tumor or a malignant cell growth, proliferation or metastasis-mediated, solid tumors and non-solid tumors such as leukemia and initiate a medical condition.

The term “treatment”, “treating” or “treated”, as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, reducing the number of tumors, preventing or delaying the development of a tumor, or some combination thereof.

The term “prevent”, “preventing”, or “prevention”, as used herein in the context of preventing a condition, refers generally to preventing or delaying the onset of the disease, or preventing the manifestation of clinical or subclinical symptoms thereof in a subject (whether a human or animal), for example, preventing the disease from occurring in a subject predisposed to the condition or disease but has not yet been diagnosed as having it.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. Specifically, the “therapeutically-effective amount,” refers to an antibody in an amount or concentration effective to treat the human CD47/HER2-related diseases or conditions.

The present disclosure in a “host cell”, as used herein, refers to a cell with the introduction of exogenous polynucleotides.

The term “pharmaceutically acceptable”, as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro AR, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), Coryne bacterium parvum, lipopolysaccharide, cytokines, and the like. Freund's adjuvant is the most commonly used adjuvant in animal experiments now. Aluminum hydroxide adjuvant is more commonly used in clinical trials.

### Bispecific Polypeptide Complexes

The bispecific polypeptide complexes provided herein include bispecific antibodies and antigen-binding portions thereof. As used herein, the term “polypeptide complex” can be used interchangeably with “antibody”. In some embodiments, the bispecific antibodies and antigen-binding portions thereof have a first specificity for HER2 (e.g., human, cynomolgus monkey and mouse HER2), and a second specificity for CD47 (e.g., human, cynomolgus monkey and mouse CD47). Such antibodies may be referred to herein as, e.g., “anti-HER2/anti-CD47,” or “anti-CD47/HER2,” or “anti-CD47×HER2” or “CD47×HER2” bispecific antibodies, or other similar terminology.

In some embodiments, the bispecific antibodies herein comprise a first antigen-binding moiety that specifically binds to CD47 (CD47-binding moiety) and a second antigen-binding moiety that specifically binds to HER2 (HER2-binding moiety). The first and second antigen-binding moiety may be in Fab, scFv, or VHH format etc., considering the stability, expression level, binding capacity and other functions of the assembled antibody. For example, the HER2-binding moiety is in Fab, scFv, or VHH format, and the CD47-binding moiety is in Fab format; alternatively, the CD47-binding moiety is in Fab, scFv, or VHH format, and the HER2-binding moiety is in Fab format. In some embodiments, both the HER2-binding moiety and the CD47-binding moiety are in Fab format, forming two arms of the bispecific antibody.

In some embodiments, the bispecific antibodies as disclosed herein comprise more than one antigen-binding moieties that specifically bind to CD47 and/or more than one antigen-binding moieties that specifically binds to HER2. Usually, for a bispecific antibody, said more than one antigen-binding moieties have the same variable regions (thus targeting the same antigen/epitope), or are completely the same in the variable regions and constant regions (if present). For example, the antibodies may comprise two same CD47-binding moieties and one HER2-binding moiety, or one CD47-binding moiety and two same HER2-binding moieties, or two same CD47-binding moieties and two same HER2-binding moieties. In addition, where two CD47-binding moieties or two HER2-binding moieties are present, preferably the two CD47-binding moieties and/or the two HER2-binding moieties are not on the same chains.

In some specific embodiments, the CD47-binding moiety is in Fab format comprising a first VH operably linked to an antibody heavy chain CH1 domain, and a first VL operably linked to an antibody light chain constant (CL) domain, and the HER2-binding moiety is also in Fab format but comprises a second heavy chain variable domain (VH) operably linked to a first T cell receptor (TCR) constant region (C1), and a second light chain variable domain (VL) operably linked to a second TCR constant region (C2). In other words, in the second antigen-binding moiety, the commonly adopted CH1 domain and CL domain are replaced by a pair of TCR constant regions. The positions of C1 and C2 can be exchanged.

Alternatively, the CD47-binding moiety may comprise a first heavy chain variable domain (VH) operably linked to a first T cell receptor (TCR) constant region (C1), and a first light chain variable domain (VL) operably linked to a second TCR constant region (C2), wherein the positions of C1 and C2 can be exchanged, while the HER2-binding moiety may comprise a second VH operably linked to an antibody heavy chain CH1 domain, and a second VL operably linked to an antibody light chain constant (CL) domain.

The introduction of TCR constant regions to replace the commonly used CH1 and CL domains have been shown to increase the stability and expression level of the generated antibody format. A detailed description of the utility of TCR constant regions in assembling two parental antibodies into a bispecific molecule with desired valency and functionality have been disclosed in WO2019/057122, the full content of which is herein incorporated by reference. The replacement by TCR constant region (CBeta/CAlpha) results a chimeric Fab that possesses a unique light-heavy chain interface orthogonal to that of a regular antibody Fab. The assembly of the chimeric and regular Fabs in different formats can create various bispecific molecules with different structures and valences.

The first TCR constant region and the second TCR constant region are associated via a non-native interchain disulfide bond. The pair of TCR constant regions in the antigen-binding moiety includes TCR alpha and beta constant regions (wild-type or preferably engineered) in the light chain and heavy chain respectively. The TCR constant regions in the bispecific antibodies are capable of associating with each other to form a dimer through a non-native disulfide bond.

The TCR, i.e., T cell receptor, is a heterodimeric T cell surface protein that belongs to the immunoglobulin superfamily and is similar to a half antibody with a single heavy chain and a single light chain. A native TCR has an extracellular portion, a transmembrane portion, and an intracellular portion. The extracellular domain of a TCR has a membrane-proximal constant region and a membrane-distal variable region.

The sequences of constant regions of wild type human TCR beta and alpha chain can be found in NCBI accession number A0A5B9 (www.uniprot.org/uniprot/A0A5B9) and NCBI accession number P01848 (www.uniprot.org/uniprot/P01848). The pair of TCR constant regions for constructing the bispecific antibodies herein are derived from the wild type TCR constant regions, with one or more substitutions, additions or deletions of one or more amino acids.

As illustrated in the present application, the bispecific antibody comprises an engineered TCR beta chain constant region, the sequence as shown in SEQ ID NO: 17; and an engineered TCR alpha chain constant region, the sequence as shown in SEQ ID NO: 18.

It is understood the variants of TCR constant regions are not limited to the above sequences, as long as they can stabilize the VH and VL regions to form the antigen-binding moiety. Multiple C1 and C2 variants for constructing WuXiBody antibody formats have been disclosed in PCT/CN2021/072601, the full content of which is incorporated herein by reference.

In some embodiments, the TCR beta chain constant region replaces the CH1 domain and TCR alpha chain constant region replace the CL domain. Alternatively, they may be exchanged, with TCR beta chain constant region in the light chain and TCR alpha chain constant region in the heavy chain.

The benefits provided by replacing CH1 and CL domains with TCR constant regions is significant. In the bispecific antibody, the C1 and C2 comprising antigen-binding moiety with at least one non-native disulfide bond can be recombinantly expressed and assembled into the desired conformation, which stabilizes the TCR constant region dimer while providing for good antigen-binding activity of the antibody variable regions. Moreover, the C1 and C2 comprising antigen-binding moiety is found to well tolerate routine antibody engineering, for example, modification of glycosylation sites, and removal of some natural sequences. Furthermore, the bispecific antibodies in such format can be readily expressed and assembled with minimal or substantially no mispairing of the antigen-binding sequences due to the presence of the TCR constant regions in the antigen-binding moiety.

In some specific embodiments, the bispecific antibodies as disclosed herein comprises one HER2-binding moiety and one CD47-binding moiety in each arm, i.e. comprising two HER2-binding moieties and two CD47-binding moieties per antibody. The Fc region may be located at the C terminal of the antibodies, operably linking to the CD47-binding moiety or the HER2-binding moiety at the N terminal of the Fc region. Alternatively, the Fc region may be located between the CD47-binding moiety and the HER2-binding moiety. Such bispecific antibodies may be constructed as a homodimer with two identical heavy chains.

According to some exemplary embodiments, the bispecific polypeptide complex herein comprises two heavy chains and four light chains, wherein from the N-terminus to the C-terminus:
(a) the first and second heavy chains each comprises domains operably linked as in VH1-C1-VH2-CH1-hinge-Fc (VH1-C1 is from the HER2-binding moiety, VH2-CH1 is from the CD47-binding moiety); two light chains comprise domains operably linked as in VL1-C2 (VL1-C2 is from the HER2-binding moiety); and the other two light chains comprise domains operably linked as in VL2-CL (VL2-CL is from the CD47-binding moiety);
(b) the first and second heavy chains each comprises domains operably linked as in VH2-CH1-VH1-C1-hinge-Fc (VH1-C1 is from the HER2-binding moiety, VH2-CH1 is from the CD47-binding moiety); two light chains comprise domains operably linked as in VL1-C2 (VL1-C2 is from the HER2-binding moiety); and the other two light chains comprise domains operably linked as in VL2-CL (VL2-CL is from the CD47-binding moiety);
(c) the first and second heavy chains each comprises domains operably linked as in VH1-C1-hinge-Fc-VH2-CH1 (VH1-C1 is from the HER2-binding moiety, VH2-CH1 is from the CD47-binding moiety); two light chains comprise domains operably linked as in VL1-C2 (VL1-C2 is from the HER2-binding moiety); and the other two light chains comprise domains operably linked as in VL2-CL (VL2-CL is from the CD47-binding moiety);
(d) the first and second heavy chains each comprises domains operably linked as in VH2-CH1-hinge-Fc-VH1-C1 (VH1-C1 is from the HER2-binding moiety, VH2-CH1 is from the CD47-binding moiety); two light chains comprise domains operably linked as in VL1-C2 (VL1-C2 is from the HER2-binding moiety); and the other two light chains comprise domains operably linked as in VL2-CL (VL2-CL is from the CD47-binding moiety);
(e) the first and second heavy chains each comprises domains operably linked as in VH1-CH1-VH2-C1-hinge-Fc (VH1-CH1 is from the HER2-binding moiety, VH2-C1 is from the CD47-binding moiety); two light chains each comprises domains operably linked as in VL1-CL (VL1-CL is from the HER2-binding moiety); and the other two light chains each comprises domains operably linked as in VL2-C2 (VL2-C2 is from the CD47-binding moiety);
(f) the first and second heavy chains each comprises domains operably linked as in VH2-C1-VH1-CH1-hinge-Fc (VH1-CH1 is from the HER2-binding moiety, VH2-C1 is from the CD47-binding moiety); two light chains each comprises domains operably linked as in VL1-CL (VL1-CL is from the HER2-binding moiety); and the other two light chains each comprises domains operably linked as in VL2-C2 (VL2-C2 is from the CD47-binding moiety);
(g) the first and second heavy chains each comprises domains operably linked as in VH1-CH1-hinge-Fc-VH2-C1 (VH1-CH1 is from the HER2-binding moiety, VH2-C1 is from the CD47-binding moiety); two light chains each comprises domains operably linked as in VL1-CL (VL1-CL is from the HER2-binding moiety); and the other two light chains each comprises domains operably linked as in VL2-C2 (VL2-C2 is from the CD47-binding moiety); or
(h) the first and second heavy chains each comprises domains operably linked as in VH2-C1-hinge-Fc-VH1-CH1 (VH1-CH1 is from the HER2-binding moiety, VH2-C1 is from the CD47-binding moiety); two light chains each comprises domains operably linked as in VL1-CL (VL1-CL is from the HER2-binding moiety); and the other two light chains each comprises domains operably linked as in VL2-C2 (VL2-C2 is from the CD47-binding moiety).

In some specific embodiments, the bispecific antibodies as disclosed herein comprises one HER2-binding moiety and two CD47-binding moieties per antibody, or two HER2-binding moieties and one CD47-binding moiety per antibody.

According to some exemplary embodiments, the bispecific polypeptide complex herein comprises two heavy chains and three light chains, wherein from the N-terminus to the C-terminus:
(a) the first heavy chain comprises domains operably linked as in VH1-C1-VH2-CH1-hinge-Fc, the second heavy chain comprises domains operably linked as in VH2-CH1-hinge-Fc; the first light chain comprises domains operably linked as in VL1-C2, and the second and third light chains comprise domains operably linked as in VL2-CL;
(b) the first heavy chain comprises domains operably linked as in VH2-CH1-hinge-Fc-VH1-C1, the second heavy chain comprises domains operably linked as in VH2-CH1-hinge-Fc; the first light chain comprises domains operably linked as in VL1-C2, and the second and third light chains comprise domains operably linked as in VL2-CL;
(c) the first heavy chain comprises domains operably linked as in VH2-CH1-VH1-C1-hinge-Fc, the second heavy chain comprises domains operably linked as in VH1-C1-hinge-Fc; the first and second light chains each comprises domains operably linked as in VL1-C2, and the third light chain comprises domains operably linked as in VL2-CL;
(d) the first heavy chain comprises domains operably linked as in VH1-C1-hinge-Fc-VH2-CH1, the second heavy chain comprises domains operably linked as in VH1-C1-hinge-Fe; the first and second light chains each comprises domains operably linked as in VL1-C2, and the third light chain comprises domains operably linked as in VL2-CL;
(e) the first heavy chain comprises domains operably linked as in VH1-CH1-VH2-C1-hinge-Fe, the second heavy chain comprises domains operably linked as in VH2-C1-hinge-Fc; the first light chain comprises domains operably linked as in VL1-CL, and the second and third light chains comprise domains operably linked as in VL2-C2;
(f) the first heavy chain comprises domains operably linked as in VH2-C1-hinge-Fc-VH1-CH1, the second heavy chain comprises domains operably linked as in VH2-C1-hinge-Fe; the first light chain comprises domains operably linked as in VL1-CL, and the second and third light chains comprise domains operably linked as in VL2-C2;
(g) the first heavy chain comprises domains operably linked as in VH2-C1-VH1-CH1-hinge-Fc, the second heavy chain comprises domains operably linked as in VH1-CH1-hinge-Fe, the first and second light chains each comprises domains operably linked as in VL1-CL, and the third light chain comprises domains operably linked as in VL2-C2; or
(h) the first heavy chain comprises domains operably linked as in VH1-CH1-hinge-Fc-VH2-C1, the second heavy chain comprises domains operably linked as in VH1-CH1-hinge-Fe, the first and second light chains each comprises domains operably linked as in VL1-CL, and the third light chain comprises domains operably linked as in VL2-C2.

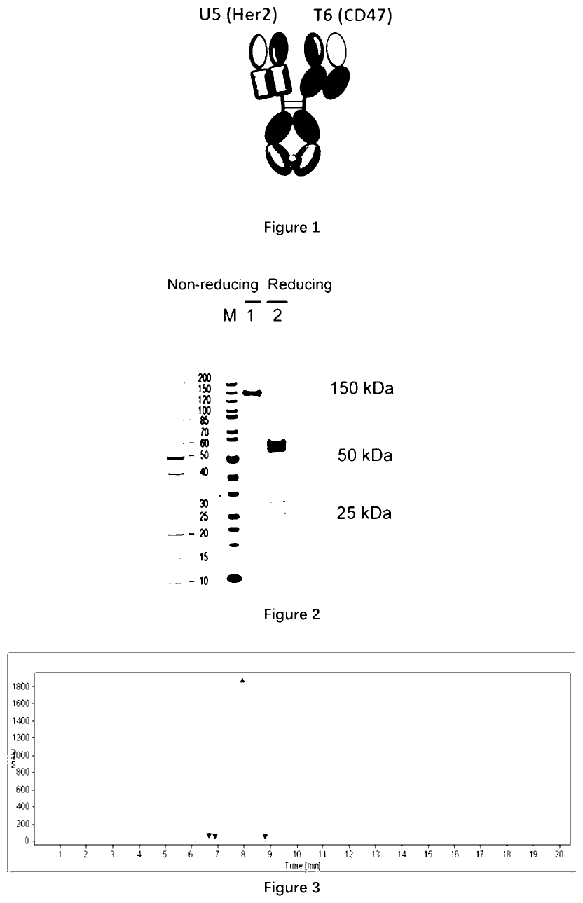

In some specific embodiments, the bispecific antibodies as disclosed herein comprises one HER2-binding moiety in one arm and one CD47-binding moiety in the other arm. Depicted in FIG. 1 is a bispecific antibody comprising the following structure (E17): from N terminus to C terminus, in the first heavy chain, VH1-C1-hinge-Fc; in the second heavy chain, VH2-CH1-hinge-Fc; in the first light chain, VL1-C2; and in the second light chain, VL2-CL. VH1 and VL1 refer to the first VH and VL that form a HER2 binding site, and VH2 and VL2 refer to the second VH and VL that form a CD47 binding site, respectively. “-” represents an operable linkage, generally via a linker comprising a peptide bond or a peptide linker with about 1-30 amino acids. The peptide linker may be a series of (G4S)n, with n=1-5.

Depending on the bispecific format and/or numbering convenience, the numbering sequences as disclosed herein, such as first or second, could be different. For example, the first VH may be numbered as the second VH, and the first VL may be numbered as the second VL. As another example, the second antigen-binding moiety may be numbered as the first antigen-binding moiety, and the first antigen-binding moiety may be numbered as the second antigen-binding moiety, depending on preference and/or convenience.

### CDRs and Variable Regions of the Bispecific Polypeptide Complex

In some embodiments, the bispecific polypeptide complex or antigen-binding portion thereof comprises a first antigen-binding moiety that specifically binds to HER2 (preferably human HER2) and a second antigen-binding moiety that specifically binds to CD47 (preferably human CD47), wherein the first and second antigen-binding moieties are derived from an anti-CD47 antibody and anti-HER2 antibody, respectively. The parental antibodies may be already developed and known to the public, or developed de novo. By “derived from”, it is generally meant herein that the antigen-binding moiety comprises the CDR sequences or highly homologous CDR sequences of the parent antibody, and preferably, comprises the variable regions of the parent antibody. The antigen-binding moiety may also comprise the variants of the CDR sequences of the parent antibody which retain the antigen-binding specificity. For example, compared to the original CDR sequences of parent antibody, one or two amino acids in one or more CDR regions may be modified to reduce glycosylation and deamidation risk.

Specifically, in the bispecific antibodies as exemplified herein, the HER2-binding moiety comprises:
A) one or more heavy chain CDRs (HCDRs) selected from the group consisting of
   (i) a HCDR1 comprising the amino acid sequence of SEQ ID NO: 1 or an amino acid sequence with addition, deletion and/or substitution of no more than 1, 2 or 3 amino acids compared with SEQ ID NO: 1;
   (ii) a HCDR2 comprising the amino acid sequence of SEQ ID NO: 2 or an amino acid sequence with addition, deletion and/or substitution of no more than 1, 2 or 3 amino acids compared with SEQ ID NO: 2; and
   (iii) a HCDR3 comprising the amino acid sequence of SEQ ID NO: 3 or an amino acid sequence with addition, deletion and/or substitution of no more than 1, 2 or 3 amino acids compared with SEQ ID NO: 3;
B) one or more light chain CDRs (LCDRs) selected from the group consisting of:
   (i) a LCDR1 comprising the amino acid sequence of SEQ ID NO: 4 or an amino acid sequence with addition, deletion and/or substitution of no more than 1, 2 or 3 amino acids compared with SEQ ID NO: 4;
   (ii) a LCDR2 comprising the amino acid sequence of SEQ ID NO: 5 or an amino acid sequence with addition, deletion and/or substitution of no more than 1, 2 or 3 amino acids compared with SEQ ID NO: 5; and
   (iii) a LCDR3 comprising the amino acid sequence of SEQ ID NO: 6 or an amino acid sequence with addition, deletion and/or substitution of no more than 1, 2 or 3 amino acids compared with SEQ ID NO: 6; or
C) one or more HCDRs of A) and one or more LCDRs of B); and the CD47-binding moiety comprises:
A′) one or more HCDRs selected from the group consisting of:
   (i) a HCDR1 comprising the amino acid sequence of SEQ ID NO: 7 or an amino acid sequence with addition, deletion and/or substitution of no more than 1, 2 or 3 amino acids compared with SEQ ID NO: 7;
   (ii) a HCDR2 comprising the amino acid sequence of SEQ ID NO: 8 or an amino acid sequence with addition, deletion and/or substitution of no more than 1, 2 or 3 amino acids compared with SEQ ID NO: 8; and
   (iii) a HCDR3 comprising the amino acid sequence of SEQ ID NO: 9 or an amino acid sequence with addition, deletion and/or substitution of no more than 1, 2 or 3 amino acids compared with SEQ ID NO: 9;
B′) one or more LCDRs selected from the group consisting of:
   (i) a LCDR1 comprising the amino acid sequence of SEQ ID NO: 10 or an amino acid sequence with addition, deletion and/or substitution of no more than 1, 2 or 3 amino acids compared with SEQ ID NO: 10;
   (ii) a LCDR2 comprising the amino acid sequence of SEQ ID NO: 11 or an amino acid sequence with addition, deletion and/or substitution of no more than 1, 2 or 3 amino acids compared with SEQ ID NO: 11; and
   (iii) a LCDR3 comprising the amino acid sequence of SEQ ID NO: 12 or an amino acid sequence with addition, deletion and/or substitution of no more than 1, 2 or 3 amino acids compared with SEQ ID NO: 12; or
C′) one or more HCDRs of A′) and one or more LCDRs of B′).

Variable regions and CDRs in an antibody sequence can be identified according to general rules that have been developed in the art or by aligning the sequences against a database of known variable regions. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of any definition to refer to a CDR of the antibody as disclosed herein is intended to be within the scope of present application. The CDRs indicated in Table 2 below are defined by the Kabat and IMGT numbering system. However, the Chothia, MacCallum, and other methods known in the art could also be used to define the CDRs. Methods for identifying these regions are described in e.g., Kontermann and Dubel, eds., Antibody Engineering, Springer, New York, NY, 2001 and Dinarello et al., Current Protocols in Immunology, John Wiley and Sons Inc., Hoboken, NJ, 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein

In some specific embodiments, the HER2-binding moiety comprises a heavy chain variable region (VH) and a light chain variable region (VL), wherein
(a) the VH comprises: a HCDR1 as set forth in SEQ ID NO: 1; a HCDR2 as set forth in SEQ ID NO: 2; and a HCDR3 as set forth in SEQ ID NO: 3; and
(b) the VL comprises: a LCDR1 as set forth in SEQ ID NO: 4; a LCDR2 as set forth in SEQ ID NO: 5; and a LCDR3 as set forth in SEQ ID NO: 6.

In some specific embodiments, the CD47-binding moiety comprises a heavy chain variable region (VH) and a light chain variable region (VL), wherein
(a) the VH comprises: a HCDR1 as set forth in SEQ ID NO: 7; a HCDR2 as set forth in SEQ ID NO: 8; and a HCDR3 as set forth in SEQ ID NO: 9; and
(b) the VL comprises: a LCDR1 as set forth in SEQ ID NO: 10; a LCDR2 as set forth in SEQ ID NO: 11; and a LCDR3 as set forth in SEQ ID NO: 12.

In some embodiments, the bispecific antibody or antigen-binding portion thereof comprises a first antigen-binding moiety that specifically binds to HER2, wherein the first antigen-binding moiety comprises:
(A) a heavy chain variable region:
(i) comprising the amino acid sequence of SEQ ID NO: 13;
(ii) comprising an amino acid sequence at least 85%, 90%, or 95% identical (preferably, at least 90%, more preferably, at least 95% (e.g., 95%, 96%, 97%, 98%, or 99%)) to SEQ ID NO: 13; or
(iii) comprising an amino acid sequence with addition, deletion and/or substitution of one or more (e.g., one, two, three, or more, preferably one, two, or three, more preferably, one or two) amino acids compared with SEQ ID NO: 13; and/or
(B) a light chain variable region:
(i) comprising the amino acid sequence of SEQ ID NO: 14;
(ii) comprising an amino acid sequence at least 85%, at least 90%, or at least 95% identical (preferably, at least 90%, more preferably, at least 95% (e.g., 95%, 96%, 97%, 98%, or 99%) to SEQ ID NO: 14; or
(iii) comprising an amino acid sequence with addition, deletion and/or substitution of one or more (e.g., one, two, three, or more, preferably one, two, or three, more preferably, one or two) amino acids compared with SEQ ID NO: 14.

In some further embodiments, the bispecific antibody or antigen-binding portion thereof comprises a second antigen-binding moiety that specifically binds to CD47, wherein the second antigen-binding moiety comprises:
(A) a heavy chain variable region:
(i) comprising the amino acid sequence of SEQ ID NO: 15;
(ii) comprising an amino acid sequence at least 85%, 90%, or 95% identical (preferably, at least 90%, more preferably, at least 95% (e.g., 95%, 96%, 97%, 98%, or 99%)) to SEQ ID NO: 15; or
(iii) comprising an amino acid sequence with addition, deletion and/or substitution of one or more (e.g., one, two, three, or more, preferably one, two, or three, more preferably, one or two) amino acids compared with SEQ ID NO: 15; and/or
(B) a light chain variable region:
(i) comprising the amino acid sequence of SEQ ID NO: 16;
(ii) comprising an amino acid sequence at least 85%, at least 90%, or at least 95% identical (preferably, at least 90%, more preferably, at least 95% (e.g., 95%, 96%, 97%, 98%, or 99%)) to SEQ ID NO: 16; or
(iii) comprising an amino acid sequence with addition, deletion and/or substitution of one or more (e.g., one, two, three, or more, preferably one, two, or three, more preferably, one or two) amino acids compared with SEQ ID NO: 16.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. MoI. Biol. 215:403-10. BLAST protein searches can be performed with the XBLAST program, score=50, wordlength=3 to obtain amino acid sequences homologous to the antibody molecules of the present disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

In some specific embodiments, the heavy chain variable region of the HER2-binding moiety is consisted of the amino acid sequence of SEQ ID NO: 13, and the light chain variable region of the HER2-binding moiety is consisted of the amino acid sequence of SEQ ID NO: 14, and/or the heavy chain variable region of the CD47-binding moiety is consisted of the amino acid sequence of SEQ ID NO: 15, and the light chain variable region of the CD47-binding moiety is consisted of the amino acid sequence of SEQ ID NO: 16.

In other embodiments, the amino acid sequences of the heavy chain variable region and/or the light chain variable regions can be at least 85%, 86%, 87%, 88%, 89%, 90%, 91%, 92%, 93%, 94%, 95%, 96%, 97%, 98% or 99%, preferable, at least 90%, 91%, 92%, 93%, 94%, 95%, 96%, 97%, 98% or 99%, more preferably, at least 95%, 96%, 97%, 98% or 99%, identical to the respective sequences set forth above.

In some further embodiments, the bispecific antibody or the antigen-binding portion thereof may contain conservative substitution or modification of amino acids in the variable regions of the heavy chain and/or light chain. It is understood in the art that certain conservative sequence modification can be made which do not remove antigen binding. See, e.g., Brummell et al. (1993) Biochem 32:1180-8; de Wildt et al. (1997) Prot. Eng. 10:835-41; Komissarov et al. (1997) J. Biol. Chem. 272:26864-26870; Hall et al. (1992) J. Immunol. 149:1605-12; Kelley and O' Connell (1993) Biochem. 32:6862-35; Adib-Conquy et al. (1998) Int. Immunol. 10:341-6 and Beers et al. (2000) Clin. Can. Res. 6:2835-43.

As described above, the term “conservative substitution,” as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

### Antigen Binding of the Bispecific Polypeptide Complex

Preferably, the bispecific polypeptide complex of the disclosure is capable of binding to human HER2 and CD47. The binding of an antibody to HER2 and CD47 can be assessed using one or more techniques well established in the art, for instance, ELISA or FACS, which measures binding of the antibody to soluble HER2/CD47 protein or HER2/CD47 protein expressed on cell surfaces, respectively. For example, an antibody can be tested by a flow cytometry assay in which the antibody is reacted with a cell line that expresses human HER2 or human CD47, such as CHO cells that have been transfected to express HER2 or CD47 on their cell surface, or a HER2 or CD47 positive cell line, or a HER2 and CD47 double positive cell line.

Additionally or alternatively, the binding of the antibody, including the binding kinetics (e.g., K_{D} value) can be tested in BIAcore binding assays. For instance, an antibody of the disclosure binds to a human HER2 protein with a K_{D} of 1×10⁻⁹ M or less; binds to a human HER2 protein with a K_{D} of 8×10⁻¹⁰ M or less; binds to a human HER2 protein with a K_{D} of 6×10⁻¹⁰ M or less; binds to a human HER2 protein with a K_{D} of 4×10⁻¹⁰ M or less; binds to a human HER2 protein with a K_{D} of 2×10⁻¹⁰ M or less; or binds to a human HER2 protein with a K_{D} of 1.5×10⁻¹⁰ M or less, as measured by Surface Plasmon Resonance.

### Functionality of the BsAbs

The bispecific antibodies as disclosed herein are characterized by particular functional features or properties. In some embodiments, the antibodies have one or more of the following properties:
(a) specifically bind to HER2 with a high affinity, and specifically bind to CD47 with a relatively lower affinity compared to a control antibody;
(b) bind to target cells without being affected by an antigen sink effect (e.g., of human blood cells or Jurkat cells);
(c) have negligible binding to red blood cells (RBCs), avoiding inducing hemagglutination and phagocytosis of human RBCs;
(d) have greatly reduced (e.g., ˜200 fold decreased) binding to Jurkat cells compared to a control antibody;
(e) efficiently block the binding of CD47 to SIRPα on CD47/HER2 double positive cells, without being affected by antigen sink effect (e.g., of human blood cells or Jurkat cells);
(f) have almost no CD47 ligand blocking effect on CD47 single positive cells;
(g) have significantly higher CD47 occupancy on CD47/HER2 double positive cells than the control antibody;
(h) have significantly increased ADCP efficacy on CD47/HER2 double positive cells compared to a combination of anti-CD47 and anti-HER2 monotherapies, while show almost no ADCP efficacy on human blood cells;
(i) have potent ADCC efficacy on CD47/HER2 double positive cells, while showing weak ADCC efficacy on Jurkat cells;
(j) have a potent inhibitory effect on proliferation of CD47/HER2 double positive cells;
(k) significantly inhibit tumor cell growth in a HER2 positive cancer model; and
(l) have better or comparable rodent pharmacokinetics with a control antibody.

In some embodiments, the control antibody is a monoclonal antibody. In some embodiments, the control antibody is an anti-CD47 antibody. In some embodiments, the control antibody is a monoclonal anti-CD47 antibody such as Magrolimab. In some embodiments, the control antibody is an anti-HER2 antibody. In some embodiments, the control antibody is a monoclonal anti-HER2 antibody such as Trastuzumab or Panitumumab.

In some embodiments, the bispecific polypeptide complexes as disclosed herein have a higher binding affinity to HER2 as compared to monospecific anti-HER2 antibodies or other anti-HER2/CD47 bispecific antibodies. In some embodiments, the bispecific polypeptide complexes as disclosed herein have binding affinity to HER2 that is at least 10%, 20%, 30%, 40%, 50%, 60%, 70%, 80%, 90%, or 100% higher than monospecific anti-HER2 antibodies or other anti-HER2/CD47 bispecific antibodies, as measured in K_{D}.

In some embodiments, the bispecific polypeptide complexes as disclosed herein have a lower binding affinity to CD47 as compared to monospecific anti-CD47 antibodies or other anti-HER2/CD47 bispecific antibodies. In some embodiments, the bispecific polypeptide complexes as disclosed herein have binding affinity to CD47 that is at least 10%, 20%, 30%, 40%, 50%, 60%, 70%, 80%, 90%, or 100% lower than monospecific anti-CD47 antibodies or other anti-HER2/CD47 bispecific antibodies, as measured in K_{D}.

In some embodiments, the bispecific polypeptide complexes as disclosed herein have higher binding affinity to HER2 as compared to monospecific anti-HER2 antibodies and/or lower binding affinity to CD47 as compared to monospecific anti-CD47 antibodies. In some embodiments, the bispecific polypeptide complexes as disclosed herein have a higher or comparable binding affinity to HER2 as compared to Trastuzumab, and lower binding affinity to CD47 as compared to Magrolimab.

In some embodiments, the bispecific polypeptide complexes as disclosed herein have higher binding affinity to HER2 as compared to other anti-HER2/CD47 bispecific antibodies and/or lower binding affinity to CD47 as compared to other anti-HER2/CD47 bispecific antibodies.

### Unaffected by Antigen Sink Effect

Antibodies against cell membrane-associated antigens are usually subject to the target-mediated clearance, known as the antigen sink effect. The widespread expression of CD47 has been known to decrease the bioavailability of anti-CD47 mAbs at the tumor due to antigen sink effects, and also has risks of off-target toxicities, most substantially from cross-linking of red blood cells that show high expression of CD47.

Expression of CD47 on normal tissues may create an ‘antigen sink’ that prevents anti-CD47 therapeutic antibodies from reaching intended tumor cell targets in vivo. As demonstrated herein, one strategy that may circumvent this issue is to employ BsAbs with a reduced affinity for CD47. These BsAbs retain the ability to block the CD47-SIRPα interaction, but require binding to a second tumor antigen for high affinity binding.

The BsAbs disclosed herein show that targeting CD47 and HER2 could direct CD47-SIRPα blockade specifically to cells that co-express HER2, and the BsAbs exhibit therapeutic synergy not observed with monospecific anti-CD47 antibody or monospecifici anti-HER2 antibody. The BsAbs herein specific for CD47 (with reduced affinity) and HER2 (with high affinity) show negligible RBC antigen sink, and exhibit binding specificity and potent functional effects on CD47 and HER2 double positive cancer cells.

### Blocking the Binding of CD47 to SIRPα

CD47 is usually expressed on the surface of normal healthy cells and migrates hematopoietic stem cells to prevent phagocytosis, and is upregulated in nearly all hematological and solid tumors to evade immune surveillance and escape phagocytosis. Disrupting the interaction between CD47 and SIRPα enables phagocytes to “eat” and destroy cancer cells. CD47 blockade repolarizes tumor-associated macrophages into a pro-inflammatory, anti-tumor state, and clearance of malignant cells by phagocytic cells offers an additional route for neoantigen presentation to adaptive immune system.

Signal regulatory protein alpha (SIRPα, also known as CD172a) is a receptor for CD47 and mainly expresses on the surface of macrophages. CD47 is known to interact with SIRPα, and thus can escape immune surveillance. The antibodies of the present disclosure may modulate, e.g., block, inhibit, reduce, antagonize, neutralize or otherwise interfere with the binding of CD47 to SIRPα. Blockade of the CD47-SIRPα interaction by using the antibody as described herein may ameliorate or overcome the immune escape, leading to potential clinical benefits.

As demonstrated in the examples, the bispecific antibodies described herein may effectively block CD47 ligand binding on CD47/HER2 double positive cell lines, i.e., blocking the interaction of CD47 to SIRPα, whereas showing no CD47 ligand blocking on CD47 single positive cell line (e.g., Jurkat cells). Moreover, the blocking effect on CD47/HER2 double positive cell lines was not affected by antigen sink effect caused by e.g., human blood cells or Jurkat cells.

### Weak Binding to Human Red Blood Cells (RBCs) to Avoid Hemagglutination

The ubiquitous expression of CD47, especially on RBCs, limits the usage of anti-CD47 antibody therapies. Many anti-CD47 antibodies have been reported to cause hemagglutination of human red blood cells. In preclinical studies, transient hemolytic anemia was associated with anti-CD47 therapy due to elevated RBC clearance.

The antibodies of the present disclosure show negligible binding to human red blood cells and avoid the undesirable effects of hemagglutination. Compared to the control anti-CD47 antibody, the bispecific antibodies herein also show about 200-fold decreased binding on Jurkat cells. Consistently, the phagocytosis against human RBCs induced by the bispecific antibodies herein would be much milder than control antibodies.

### Mediation of ADCP or ADCC Activity

The term “antibody-dependent cellular phagocytosis” or “ADCP” is a cellular process by which effector cells with phagocytic potential, such as monocytes and macrophages, can internalize target cells. Once phagocytosed, the target cell resides in a phagosome, which fuses with a lysosome to begin degradation of the target cell via an oxygen-dependent or independent mechanism. This function is dependent on opsonization, or identification of the target cell with an antibody, which then also serves as a bridge between the target cell and the phagocytic cell. Mechanistically, the antibody binds its cognate antigen on the target cell through its antigen recognition domain, and then recruits the phagocytic cell to the target with its Fc region. Once bound to the Fc receptor of the phagocytic cell, the target cell is ingested and degraded. This process also leads to the production of soluble factors by the effector cells that help initiate and drive the immune response.

The term “antibody-dependent cell-mediated cytotoxicity” or “ADCC”, as used herein, refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

As disclosed herein, ADCP may be a mechanism of action for anti-CD47 therapeutic antibodies. The bispecific antibodies herein show a potent ADCP efficacy on CD47/HER2 double positive cells. Moreover, these antibodies of the present disclosure could induce a potent ADCC activity against CD47/HER2 double positive tumor cells. On the other hand, the BsAbs have little or very weak ADCP or ADCC efficacy on CD47 single positive cells, indicating that binding to a HER2 is necessary for the effector functions of these antibodies herein.

### Synergic Effect

The bispecific antibodies as disclosed herein, which combine CD47 and HER2 dual binding activity, exhibit a synergic effect compared to monotherapies such as Trastuzumab, Panitumumab or Magrolimab alone.

The present disclosure has demonstrated that in the HCC1954 breast cancer model or NCI-N87 xenograft model in mice, the administration of the bispecific antibody as disclosed herein achieved a significantly improved tumor growth inhibition compared to anti-HER2 mAb or anti-CD47 mAb alone. The combination of the bispecific antibody as disclosed herein with another anti-HER2 mAb further improves the anti-tumor effect at the same dosage level.

### Fc Region

The Fc region of the bispecific antibodies disclosed herein is preferably a human IgG Fc region. The IgG Fc region may be of any isotype, including, but not limited to, IgG1, IgG2, IgG3 or IgG4. In certain embodiments, the Fc region is of the IgG1 isotype.

In the context of bispecific antibodies of the present disclosure, the Fc region may comprise one or more amino acid changes (e.g., insertions, deletions or substitutions) as compared to wild-type Fc region. The disclosure encompasses bispecific antigen-binding molecules comprising one or more modifications in the Fc region to obtain the desired functionality, e.g., a “knob into hole” structure to promote heterodimerization, or a modified Fc region to change the binding interaction between Fc and FcRn or FcγR.

The term “knob into hole”, as used herein, refers to engineering the CH3 domain of antibody Fc region to create either a “knob” or a “hole” in each heavy chain to promote heterodimerization. A knob can be obtained by replacement of a small amino acid residue with a larger one in the first CH2/CH3 polypeptide, and a hole can be obtained by replacement of a large residue with a smaller one. For details of the mutation sites for knobs into holes please see Spiess et al., 2015, supra and Brinkmann et al., 2017, supra; US patent application US2003078385A1. Generally, a “knob” is created by replacing T366 with a bulky residue W on one heavy chain, and the corresponding “hole” is made by triple mutations of T366S, L368A and Y407V on the other heavy chain, according to EU numbering as in Kabat et al. In some embodiments, a “hole” mutation is Y349C, T366S, L368A, and/or Y407V, and a “knob” mutation is S354C and/or T366W. In some embodiments, the heavy chain of the bispecific antibody comprising C1 has the “hole” structure, while the heavy chain comprising C2 has the “knob” structure. Alternatively, the heavy chain of the bispecific antibody comprising C1 has the “knob” structure, while the heavy chain comprising C2 has the “hole” structure.

In certain embodiments, the first heavy chain of the bispecific antibody comprises a Fc region that comprises S354C and T366W substitutions (knob), and the second heavy chain of the bispecific antibody comprises a Fc region of IgG1 isotype that comprises Y349C, T366S, L368A and Y407V substitutions (hole). In some other embodiments, the first heavy chain of the bispecific antibody comprises a Fc region of IgG4 isotype, wherein the Fc region comprises S354C and T366W substitutions (knob), and the second heavy chain of the bispecific antibody comprises a Fc region of IgG4 isotype, wherein the Fc region comprises Y349C, T366S, L368A and Y407V substitutions (hole).

In addition, the Fc region may comprise one or more amino acid modification (e.g., Leu234Ala/Leu235Ala or LALA) that alters the antibody-dependent cellular cytotoxicity (ADCC) or other effector functions. In certain embodiments, the Fc modification comprise a LALA mutation, i.e., mutations of L234A and L235A, according to EU numbering as in Kabat et al. LALA mutation is perhaps the most commonly used mutation for disrupting antibody effector function, e.g., eliminate Fc binding to specific FcγRs, reduce ADCC activity mediated by PBMCs and monocytes. Additionally, it has been found that therapeutic potential may be enhanced by the introduction of YTE (M252Y/S254T/T256E) and LS (M428L/N434S) in the Fc regions, as a consequence of increased half-lives and prolonged duration of protection. The S228P mutation has also been found to prevent in vivo and in vitro IgG4 Fab-arm exchange as demonstrated using a combination of novel quantitative immunoassays and physiological matrix preparation (J Biol Chem 2015 Feb. 27; 290(9): 5462-9).

The bispecific antibodies as disclosed herein may comprise a Fc region selected from one of the following:
(a) a human IgG1 Fc region engineered to comprise a “knob into hole” structure;
(b) a human IgG4 Fc region engineered to comprise a “knob into hole” structure and optionally a S228P mutation; and
(c) a human IgG4 Fc region engineered to comprise a S228P mutation and optionally M252Y/S254T/T256E mutations.

The “EU numbering system” or “EU index” is generally used when referring to a residue in an immunoglobulin heavy chain constant region (e.g., the EU index reported in Kabat et al., supra). The “EU numbering as in Kabat” or “EU index as in Kabat” refers to the residue numbering of the human IgG1 EU antibody. Unless stated otherwise herein, references to residue numbers in the constant domain of antibodies means residue numbering by the EU numbering system.

### Nucleic Acid Molecules Encoding the BsAbs

In some aspects, the disclosure is directed to an isolated nucleic acid molecule, comprising a nucleic acid sequence encoding one or more chains of the bispecific antibodies or antigen-binding portion thereof.

Nucleic acids of the present disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

The isolated nucleic acid encoding the VH region can be converted to a full-length heavy chain gene by operatively linking the VH-encoding nucleic acid to another DNA molecule encoding heavy chain constant regions (CH1, CH2 and CH3, or TCR beta constant region). The sequences of human heavy chain constant region genes are known in the art (see e.g., Kabat et al. (1991), supra) and DNA fragments encompassing these regions can be obtained by standard PCR amplification. The heavy chain constant region can be an IgG1, IgG2, IgG3, IgG4, IgA, IgE, IgM or IgD constant region, but more preferably is an IgG1 or IgG4 constant region.

The isolated nucleic acid encoding the VL region can be converted to a full-length light chain gene (as well as a Fab light chain gene) by operatively linking the VL-encoding DNA to another DNA molecule encoding the light chain constant region, CL, or TCR alpha constant region. The sequences of human light chain constant region genes are known in the art (see e.g., Kabat et al., supra) and DNA fragments encompassing these regions can be obtained by standard PCR amplification. In some embodiments, the light chain constant region can be a kappa or lambda constant region.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

In some specific embodiments, the isolated nucleic acid molecule comprises one or more nucleic acid sequence(s) selected from the group consisting of:
(A) a nucleic acid sequence that encodes the heavy chain sequence of the CD47-binding moiety or the heavy chain sequence of the HER2-binding moiety;
(B) a nucleic acid sequence that encodes the heavy chain sequence of the CD47-binding moiety operably linked to the heavy chain sequence of the HER2-binding moiety;
(C) a nucleic acid sequence that encodes the light chain sequence of the CD47-binding moiety;
(D) a nucleic acid sequence that encodes a light chain sequence of the HER2-binding moiety;
(E) any combinations of (A)-(D); and
(F) a nucleic acid sequence that hybridized under high stringency conditions to the complementary strand of the nucleic acid sequence of (A)-(E).

In some specific embodiments, the isolated nucleic acid molecule comprises a nucleic acid sequence encoding SEQ ID NO: 19. In some specific embodiments, the isolated nucleic acid molecule comprises a nucleic acid sequence encoding SEQ ID NO: 20. In some specific embodiments, the isolated nucleic acid molecule comprises a nucleic acid sequence encoding SEQ ID NO: 21. In some specific embodiments, the isolated nucleic acid molecule comprises a nucleic acid sequence encoding SEQ ID NO: 22.

Exemplary high stringency conditions include hybridization at 45° C. in 5×SSPE and 45% formamide, and a final wash at 65° C. in 0.1×SSC. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al, (Eds.), Molecular Cloning: A laboratory Manual. Cold Spring Harbor Laboratory Press: Cold Spring Harbor, New York (1989), pp. 9.47 to 9.51.

### Vectors and Host Cells

The nucleic acid molecules that encodes the bispecific polypeptide complexes can be inserted into a vector for further cloning (amplification of the DNA) or for expression, using recombinant techniques known in the art. In some embodiments, the antibody may be produced by homologous recombination known in the art. DNA encoding the monoclonal antibody is readily isolated and sequenced using conventional procedures (e.g., by using oligonucleotide probes that are capable of binding specifically to genes encoding the heavy chain of the antibody). Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Selectable marker genes may include the dihydrofolate reductase (DHFR) gene (for use in dhfr-host cells with methotrexate selection/amplification) and the neo gene (for G418 selection).

In some embodiments, the vector system includes mammalian, bacterial, yeast systems, etc, and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS420, pLexA, pACT2.2 etc, and other laboratorial and commercially available vectors. Suitable vectors may include, plasmid, or viral vectors (e.g., replication defective retroviruses, adenoviruses and adeno-associated viruses). In one embodiment of the disclosure, the vector may be pET, for instance, pETbac containing genes of hexa-histidine- and c-Myc-tag.

Vectors comprising the nucleic acid sequence encoding the bispecific polypeptide complexes can be introduced to a host cell for cloning or gene expression. Thus, the present disclosure also relates to a recombinant eukaryotic or prokaryotic host cell which produces a bispecific polypeptide complex of the present disclosure, such as a transfectoma.

Suitable host cells for cloning or expressing the DNA in the vectors herein are the prokaryote, yeast, or higher eukaryote cells, such as mammalian cells. Mammalian host cells for expressing the antibodies of the present disclosure include Chinese Hamster Ovary (CHO cells) (including dhfr CHO cells, described in Urlaub and Chasin, (1980) Proc. Natl. Acad. ScL USA 77:4216-4220, used with a DHFR selectable marker, e.g., as described in R. J. Kaufman and P. A. Sharp (1982) J. MoI. Biol. 159:601-621), COS cells and SP2 cells. In particular, for use with NSO myeloma cells, another expression system is the GS gene expression system disclosed in WO 87/04462, WO 89/01036 and EP 338,841. Also included are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., 1980, Proc. Natl. Acad. Sci. USA 77:4216); mouse sertoli cells (TM4, Mather, 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; mouse myeloma cells, such as NSO (e.g. RCB0213, 1992, Bio/Technology 10:169) and SP2/0 cells (e.g. SP2/0-Ag14 cells, ATCC CRL 1581); rat myeloma cells, such as YB2/0 cells (e.g. YB2/3HL.P2.G11.16Ag.20 cells, ATCC CRL 1662); PER.C6 cells; and a human hepatoma line (Hep G2). CHO cells are one of the cell lines that can be used herein, with CHO-K1, DUK-B11, CHO-DP12, CHO-DG44 (Somatic Cell and Molecular Genetics 12:555 (1986)), and Lec13 being exemplary host cell lines. In the case of CHO-K1, DUK-B11, DG44 or CHO-DP12 host cells, these may be altered such that they are deficient in their ability to fucosylate proteins expressed therein.

Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for bispecific antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurosporacrassa; Schwanniomyces such as Schwanniomyces occidentalis; and filamentous fungi such as, e.g., Neurospora, Penicillium, Tolypocladium, and Aspergillus hosts such as A. nidulans and A. niger.

Other suitable host cells for the expression of the bispecific polypeptide complexes provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present disclosure, particularly for transfection of Spodoptera frugiperda cells. Plant cell cultures of cotton, corn, potato, soybean, petunia, tomato, and tobacco can also be utilized as hosts.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the bispecific polypeptide complexes provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

Following any preliminary purification step (s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Compositions

In some aspects, the disclosure is directed to a pharmaceutical composition comprising the bispecific polypeptide complexes as disclosed herein and a pharmaceutically acceptable carrier.

### Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the bispecific polypeptide complexes enhance the immune response against the vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed herein, the compositions containing the antibody include one or more anti-oxidants such as methionine, to reduce the oxidization of the antibody. The oxidation reduction may prevent or reduce a decrease in binding affinity, thereby enhancing antibody stability and extended shelf life. Thus, in some embodiments, the present disclosure provides a composition comprising one or more antibodies and one or more anti-oxidants such as methionine. The present disclosure further provides a variety of methods, wherein an antibody is mixed with one or more anti-oxidants, such as methionine, so that the antibody thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Administration, formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, i.e., dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the bispecific polypeptide complexes maybe administered in various ranges. In some embodiments, the bispecific polypeptide complexes as provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the antibody is administered at a dosage of about 50 mg/kg or less, and in certain of these embodiments the dosage is 10 mg/kg or less, 5 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

In any event, the antibody of the disclosure is preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody of the instant disclosure will comprise multiple doses of the selected drug product over a period of weeks or months. More specifically, the antibody of the instant disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection or infusion) may comprise the bispecific polypeptide complexes as provided herein in concentrations of from about 10 μg/ml to about 100 mg/ml. In some embodiments, the concentrations of the bispecific antigen binding molecule may comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments, the concentration of the bispecific antigen binding molecule comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

### Applications/Indications

The bispecific polypeptide complexes of the present disclosure have numerous in vitro and in vivo utilities. For example, these molecules can be administered to cells in culture, in vitro or ex vivo, or to human subjects, e.g., in vivo, to enhance immunity in a variety of situations. The immune response can be augmented, stimulated or up-regulated.

Preferred subjects include human patients in need of enhancement of an immune response. The methods are particularly suitable for treating human patients having a disorder that can be treated by augmenting an immune response (e.g., the T-cell mediated immune response, phagocytosis of tumor cells). The methods are particularly suitable for treatment of cancer in vivo. To achieve antigen-specific enhancement of immunity, the bispecific antibodies can be administered together with an antigen of interest or the antigen may already be present in the subject to be treated (e.g., a tumor-bearing or virus-bearing subject). When the bispecific antibodies are administered together with another agent, the two can be administered in either order or simultaneously.

### Treatment of Disorders Including Cancers

In some aspects, the present disclosure provides a method of treating a disorder in a subject, which comprises administering to the subject (for example, a human) in need of treatment a therapeutically effective amount of the antibody or antigen-binding portion thereof as disclosed herein. For example, the disorder is a cancer.

A variety of cancers where CD47 and/or HER2 is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers. In some specific embodiments, the cancer is skin cancer (such as skin squamous cell carcinoma), colon cancer, colorectal cancer (such as colorectal adenocarcinoma), lung cancer (such as lung adenocarcinoma) or breast cancer (such as triple negative breast adenocarcinoma).

In some other embodiments, the disorder is an autoimmune disease. Examples of autoimmune diseases that may be treated with the antibody or antigen-binding portion thereof include autoimmune encephalomyelitis, lupus erythematosus, and rheumatoid arthritis. The antibody or the antigen-binding portion thereof may also be used to treat or prevent infectious disease, inflammatory disease (such as allergic asthma) and chronic graft-versus-host disease.

Combined Use with Chemotherapies

The antibody may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in some embodiments selected anti-cancer agents will be linked to the unpaired cysteines of the engineered antibodies to provide engineered conjugates as set forth herein. Accordingly, such engineered conjugates are expressly contemplated as being within the scope of the instant disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In some embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleuritesfordii proteins, dianthin proteins, Phytolaccamericana proteins (PAPI, PAPII, and PAP-S), Momordicacharantia inhibitor, curcin, crotin, Saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the instant disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the site-specific constructs of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, OR), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, HER2 and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies

The present disclosure also provides for the combination of the antibody with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### Pharmaceutical Packs and Kits

Pharmaceutical packs and kits comprising one or more containers, comprising one or more doses of the antibody are also provided. In some embodiments, a unit dosage is provided wherein the unit dosage contains a predetermined amount of a composition comprising, for example, the antibody, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in some embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present disclosure also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the antibody, such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the antibody, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

Appended to the instant application is a sequence listing comprising a number of amino acid sequences. The following Table A provides a summary of the included sequences.

TABLE A

| Seq. No. | Description and sequence |
|---|---|
| 1 | Amino acid sequence of Anti-HER2 moiety HCDR1 |
| | GFNIKDTYIH |
| 2 | Amino acid sequence of Anti-HER2 moiety HCDR2 |
| | RIYPTNGYTRYADSVKG |
| 3 | Amino acid sequence of Anti-HER2 moiety HCDR3 |
| | WGGDGFYAMDY |
| 4 | Amino acid sequence of Anti-HER2 moiety LCDR1 |
| | RASQDVNTAVA |
| 5 | Amino acid sequence of Anti-HER2 moiety LCDR2 |
| | SASFLYS |
| 6 | Amino acid sequence of Anti-HER2 moiety LCDR3 |
| | QQHYTTPPT |
| 7 | Amino acid sequence of Anti-CD47 moiety HCDR1 |
| | GFTFSNFAMS |
| 8 | Amino acid sequence of Anti-CD47 moiety HCDR2 |
| | TISASGGRTFYADSVKG |
| 9 | Amino acid sequence of Anti-CD47 moiety HCDR3 |
| | EGSFGEGVDP |
| 10 | Amino acid sequence of Anti-CD47 moiety LCDR1 |
| | SGDALPKKYAY |
| 11 | Amino acid sequence of Anti-CD47 moiety LCDR2 |
| | EDNKRPS |
| 12 | Amino acid sequence of Anti-CD47 moiety LCDR3 |
| | YSTDISGNHWV |
| 13 | Amino acid sequence of Anti-HER2 moiety VH region |
| | EVQLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCSRW |
| | GGDGFYAMDYWGQGTLVTV |
| 14 | Amino acid sequence of Anti-HER2 moiety VL region |
| | DIQMTQSPSSLSASVGDRVTITCRASQDVNTAVAWYQQKPGKAPKLLIYS |
| | ASFLYSGVPSRFSGSRSGTDFTLTISSLQPEDFATYYCQQHYTTPPTFGQGT |
| | KVEIK |
| 15 | Amino acid sequence of Anti-CD47 moiety VH region |
| | EVQLLESGGGLVQPGGSLRLSCAASGFTFSNFAMSWVRQAPGKGLEWVS |
| | TISASGGRTFYADSVKGRITISRDNSKNTLFLQMNGLRAEDTAVYYCAKE |
| | GSFGEGVDPWGQGTLVTVSS |
| 16 | Amino acid sequence of Anti-CD47 moiety VL region |
| | SYEMTQPPSVSVSPGQTARITCSGDALPKKYAYWYQQKSGQAPVLVIYED |
| | NKRPSGIPERFSGSSSGTMATLTISGAQVEDEADYYCYSTDISGNHWVFG |
| | GGTELTVL |
| 17 | Amino acid sequence of TCR beta constant region (C1) |
| | LEDLKNVFPPEVAVFEPSEAEISHTQKATLVCLATGFYPDHVELSWWVNG |
| | KEVHSGVCTDPQPLKEQPALQDSRYALSSRLRVSATFWQNPRNHFRCQV |
| | QFYGLSENDEWTQDRAKPVTQIVSAEAWGR |
| 18 | Amino acid sequence of TCR alpha constant region (C2) |
| | PDIQNPDCAVYQLRDSKSSDKSVCLFTDFDSQTQVSQSKDSDVYITDKCV |
| | LDMRSMDFKSNSAVAWSQKSDFACANAFQNSIIPECTFFPS |
| 19 | Amino acid sequence of 1^{st} heavy chain of W308032-U5T6.E17- |
| | 57.uIgG1 (anti-HER2) |
| | 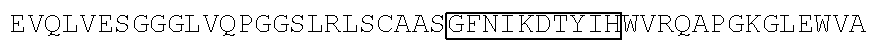 |
| | 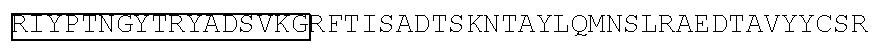 |
| | 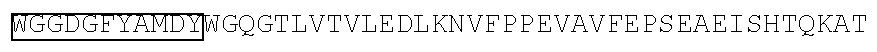 |
| | LVCLATGFYPDHVELSWWVNGKEVHSGVCTDPQPLKEQPALQDSRYALS |
| | SRLRVSATFWQNPRNHFRCQVQFYGLSENDEWTQDRAKPVTQIVSAEAW |
| | GRASDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWL |
| | NGKEYKCKVSNKALPAPIEKTISKAKGQPREPQVYTLPPCREEMTKNQVS |
| | LWCLVKGFYPSDIAVEWESNGQPENNYKTTPPVLDSDGSFFLYSKLTVDK |
| | SRWQQGNVFSCSVMHEALHNHYTQKSLSLSPG |
| 20 | Amino acid sequence of 2^{nd} heavy chain of W308032-U5T6.E17- |
| | 57.uIgG1 (anti-CD47) |
| | 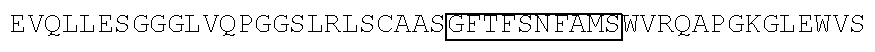 |
| | 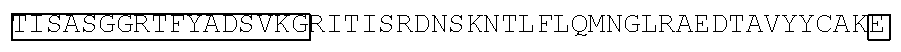 |
| | 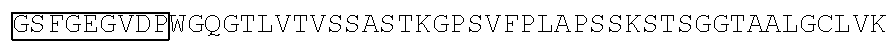 |
| | DYFPEPVTVSWNSGALTSGVHTFPAVLQSSGLYSLSSVVTVPSSSLGTQTY |
| | ICNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYN |
| | STYRVVSVLTVLHQDWLNGKEYKCKVSNKALPAPIEKTISKAKGQPREPQ |
| | VCTLPPSREEMTKNQVSLSCAVKGFYPSDIAVEWESNGQPENNYKTTPPV |
| | LDSDGSFFLVSKLTVDKSRWQQGNVFSCSVMHEALHNHYTQKSLSLSPG |
| 21 | Amino acid sequence of 1^{st} light chain of W308032-U5T6.E17- |
| | 57.uIgG1 (anti-HER2) |
| | 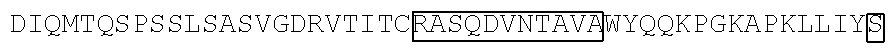 |
| | 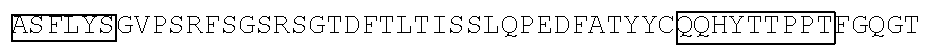 |
| | KVEIKPDIQNPDCAVYQLRDSKSSDKSVCLFTDFDSQTQVSQSKDSDVYIT |
| | DKCVLDMRSMDFKSNSAVAWSQKSDFACANAFQNSIIPECTFFPS |
| 22 | Amino acid sequence of 2^{nd} light chain of W308032-U5T6.E17- |
| | 57.uIgG1 (anti-CD47) |
| | 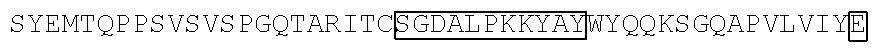 |
| | 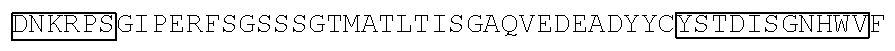 |
| | GGGTELTVLGQPKAAPSVTLFPPSSEELQANKATLVCLISDFYPGAVTVAW |
| | KADSSPVKAGVETTTPSKQSNNKYAASSYLSLTPEQWKSHKSYSCQVTH |
| | EGSTVEKTVAPTECS |

### EXAMPLES

The present disclosure, thus generally described, will be understood more readily by reference to the following Examples, which are provided by way of illustration and are not intended to be limiting of the instant disclosure. The Examples are not intended to represent that the experiments below are all or the only experiments performed.

### Example 1

### Preparation of Materials

Information on the commercially available materials used in the examples is provided in Table 1.

TABLE 1
Commercial materials

| Materials | Vendor | Cat. |
|---|---|---|
| SK-BR-3 | ATCC | HTB-30 |
| HCC1954 | ATCC | CRL-2338 |
| NCI-N87 | ATCC | CRL-5822 |
| Jurkat | ATCC | CRL-2898 |
| McCoy's 5A (1X) | Gibco | 16600-082 |
| RPMI Medium 1640 (1X) | Gibco | 22400-089 |
| RPMI Medium 1640 (1X) | Gibco | 11835-030 |
| FBS | ExCell Bio | FND500/L |
| DPBS | CORNING | 21-031-CVC |
| 0.25% Trypsin-EDTA (1X) | Gibco | 25200-072 |
| BSA | BOVOGEN | BSAS |
| PBS (10X) | Invitrogen | AM9624 |
| Expi293F ™ cells | Thermo Fisher | A14527 |
| Expi293F ™ expression medium | Thermo Fisher | A1435101 |
| ExpiFectamine293 transfection kit | Thermo Fisher | A14524 |
| Protein A column | GE Healthcare | 175438 |
| CEX column | Cytiva | 17118001 |
| HPLC-SEC | TOSOH | 0008541 |
| NuPAGE4%-12% Bis-Tris Gel | Thermo Fisher | NP0322BOX |
| Alexa647 conjugated Goat | Jackson | 109-605-098 |
| anti-Human IgG Fc | | |
| Human SIRP alpha/CD172a Protein, | Acro Biosystems | SIA-H52A8 |
| Mouse IgG1 Fc Tag | | |
| Alexa647 conjugated Goat | Jackson | 115-605-164 |
| anti-Mouse IgG Fc | | |
| Streptavidin PE | eBioscience | 12-4317-87 |
| Trastuzumab Injection | Roche | J20180073 |
| Human CD47 Protein (His & AVI | Sino Biological | 12283-H27H-B |
| Tag), Biotinylated | | |
| Human HER2/ErbB2 Protein (His | Sino Biological | 10004-H08H |
| Tag) | | |
| Series S Sensor Chip CM5 | Cytiva | 29-1496-03 |
| Anti-human IgG Fc antibody | Jackson | 109-005-098 |
| Streptavidin | Jackson | 016-000-113 |
| 10 × HBS-EP+ | Cytiva | BR100669 |
| 96-well Clear Round Bottom | Corning | #7007 |
| Ultra-Low Attachment | | |
| Versene (1X) | Life-Technology | 15040-066 |
| CD14 Microbeads, human | Miltenyi Biotec | 130-050-201 |
| rhM-CSF | R&D Systems | 216-MC |
| CellTrace ™ CFSE dye | Life-Technology | C34570 |
| CD14 Antibody, APC, Human | eBioscience | 17-0149-42 |
| EasySep ™ Human CD56 Positive | StemCell | #18055 |
| Selection Kit | | |
| Cytotoxicity Detection Kit (LDH) | Roche | 11644793001 |
| Cell Titer Glo | Promega | G7573 |
| JIMT-1 | AddexBio | CL-00917 |
| DMEM | Gibco | C11995500BT |
| FBS | Gibco | 10099-141C |
| PBS | Cytiva | SH30256.01 |
| Trypsin | Gibco | 25200-056 |

### Example 2

### Generation of W308032 Bispecific Antibodies

For BsAb generation, a CD47 binding moiety (CD47:T6) derived from an anti-CD47 antibody and a HER2 binding moiety (HER2:U5) derived from anti-HER2 antibody were combined and used for constructing a bispecific antibody.

The bispecific antibody was constructed using WuXiBody® E17 format (FIG. 1). CAlpha and CBeta genes were synthesized by Genewiz Inc. The light chain 1 was VL sequence of HER2:U5 inserted into a linearized vector containing CAlpha, and the light chain 2 was VL sequence of CD47:T6 inserted into a linearized vector containing constant region of lambda light chain. The heavy chain 1 was VH (HER2:U5)-CBeta DNA fragment inserted into a linearized vector containing human IgG1 constant region CH2-CH3 with S354C-T366W mutations and the heavy chain 2 was VH (CD47:T6) DNA fragment inserted into a linearized vector containing human IgG1 constant region CH1-CH2-CH3 with Y349C-T366S-L368A-Y407V mutations. All the sequences were cloned into modified pcDNA3.4 expression vectors.

Heavy chain and light chain expression plasmids were co-transfected into Expi293 cells using Expi293 expression system kit according to manufacturer's instructions. Five days after transfection, the supernatant of Expi293 cells expressing target proteins was collected and filtered for purification using Protein A column. Fraction from Protein A elution was collected and adjusted pH to 5.0 for IEX purification using CEX column. The CEX column was equilibrated with 50 mM NaAc, pH5.0 before and after loading. The peak fraction was collected with UA detection using 50 mM NaAc, 500 mM NaCl pH5.0 with linear step and then dialyzed in PBS buffer. The concentration of purified proteins was determined by absorbance at 280 nm. The size and purity of purified proteins were tested by SDS-PAGE and SEC-HPLC, respectively.

The obtained antibody is named as W308032-U5T6.E17-57.uIgG1 (or abbreviated as “W308032-U5T6.E17”).

TABLE 2
The CDR sequences of W308032-U5T6.E17 antibody

| | | | |
|---|---|---|---|
| Anti-HER2 | HCDR1 | HCDR2 | HCDR3 |
| moiety | SEQ ID NO: 1 | SEQ ID NO: 2 | SEQ ID NO: 3 |
| (U5) | GFNIKDTYIH | RIYPTNGYTRYADSVKG | WGGDGFYAMDY |
| | LCDR1 | LCDR2 | LCDR3 |
| | SEQ ID NO: 4 | SEQ ID NO: 5 | SEQ ID NO: 6 |
| | RASQDVNTAVA | SASFLYS | QQHYTTPPT |
| Anti-CD47 | HCDR1 | HCDR2 | HCDR3 |
| moiety | SEQ ID NO: 7 | SEQ ID NO: 8 | SEQ ID NO: 9 |
| (T6) | GFTFSNFAMS | TISASGGRTFYADSVKG | EGSFGEGVDP |
| | LCDR1 | LCDR2 | LCDR3 |
| | SEQ ID NO: 10 | SEQ ID NO: 11 | SEQ ID NO: 12 |
| | SGDALPKKYAY | EDNKRPS | YSTDISGNHWV |

### Example 3

### In Vitro Characterization of W308032 Antibodies

### 3.1 Protein Analytics

The size and purity of W308032 antibodies was evaluated by SDS-PAGE and size exclusion chromatography (SEC-HPLC). FIGS. 2 and 3 and Table 3 shows SDS-PAGE and SEC-HPLC characterizations of the W308032-U5T6.E17-57.uIgG1 after purification. FIG. 2 shows an apparent molecular mass of about 150 kDa under non-reducing condition, and about 50 kDa, 25 kDa under reducing conditions. The result indicates the bispecific molecule could assemble as expected.

TABLE 3
Purification summary

| Antibody | Purity by | Yield | Molecular | | |
|---|---|---|---|---|---|
| Name | SEC-HPLC | (mg/L) | Weight (kD) | PI | Endotoxin |
| W308032- | 96.75% | 140.53 | 147 | 6.32 | <1 EU/mg |
| U5T6.E17- | | | | | |
| 57.uIgG1 | | | | | |

### 3.2 Differential Scanning Fluorimetry (DSF)

Melting temperature (Tm) of antibodies was investigated by DSF assay using 7500 Fast Real-Time PCR system (Applied Biosystems). Briefly, 19 μL of antibody solution was mixed with 1 μl of 62.5×SYPRO Orange solution (TheromFisher-S6650) and added to a 96-well plate. The plate was heated from 26° C. to 95° C. at a rate of 2° C./min and the resulting fluorescence data was collected. Data collection and Tm calculation were automatically performed by the operating software (QuantStudio® Real Time PCR software v1.3).

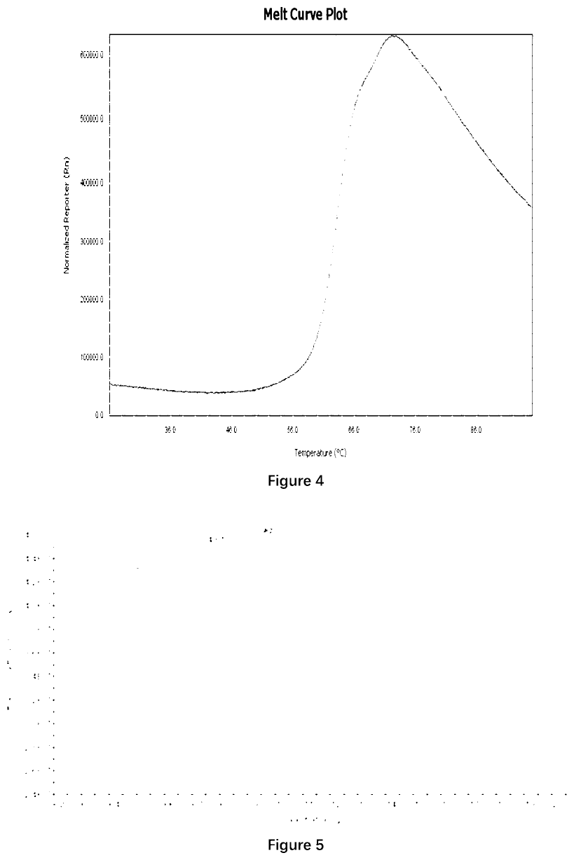

The DSF result shows Tm1 reached 63.1° C. and Tm2 reached 70.1° C., indicating W308032-U5T6.E17-57.uIgG1 has good thermal stability (FIG. 4).

TABLE 4
Summary of DSF characterization

| Antibody Name | Tm1 (° C.) | Tm2 (° C.) | Comments |
|---|---|---|---|
| W308032-U5T6.E17-57.uIgG1 | 63.1 | 70.1 | Good |

### 3.3 Diffusion Interaction Parameter (kD) Measurement by Dynamic Light Scattering (DLS)

kD measurement was investigated using DynaPro Plate Reader III (Wyatt Dynapro™). The kD parameter is the first order diffusion interaction parameter obtained in DLS assay, served as an indicator of colloidal and thermal stability of the molecule. Samples were first filtered with 0.02 μm filter and concentrated to over 20 mg/mL. Sample was diluted with PBS buffer to a final concentration at 2.5, 5, 10, 15, and 20 mg/mL. 7.5 μL sample solution was then added to 1536 well microplate. The plate was sealed with the ClearSeal Film, and centrifugated at 3,000 rpm for 5 min to let the sample down to the bottom of the well. Each sample was tested in duplicate wells. The plate was put into the corresponding position and data collection was performed by the DYNAMICS operation software (v7.8.1.3). 5 acquisitions were collected for each protein sample while each acquisition time was 5 s. For each measurement, the diffusion coefficient was determined and plotted against protein concentration. kD values were calculated automatically by the software.

The determined kD is −13.0 mL/g, indicating W308032-U5T6.E17-57.uIgG1 has good colloidal stability (FIG. 5).

TABLE 5
Summary of DLS characterization

| | | | Size | |
|---|---|---|---|---|
| Antibody Name | kD (mL/g) | R\hat{ }2 | Distribution | Comments |
| W308032-U5T6.E17- | −13.0 | 98.6 | monodisperse | Good |
| 57.uIgG1 | | | size | |

### 3.4 Hydrophobicity Interaction Chromatography HPLC (HIC-HPLC)

Hydrophobicity property of antibody was detected by HPLC 1260 Infinity II system (Agilent Technologics™) with TSKgel butyl-NPR column (Tosoh-0042168). 20 μL sample was injected into the column, and separated with a flow rate of 0.5 ml/min for 61 min. The running buffer is 25 mM sodium phosphate, pH7.0 (Buffer A) and 25 mM sodium phosphate, 1.5 M (NH4)2SO4, pH7.0 (Buffer D). The running gradient was 0% to 100% Buffe D from 3 to 53 min. The peak retention was detected with UV light of the wavelength at 280 nm and 230 nm. The retention time was analyzed with HIC-HPLC analysis method to integrate all peak areas from 20 min to 50 min. The operation and analysis software was OpenLab CDS Workstation (v2.3.0.443).

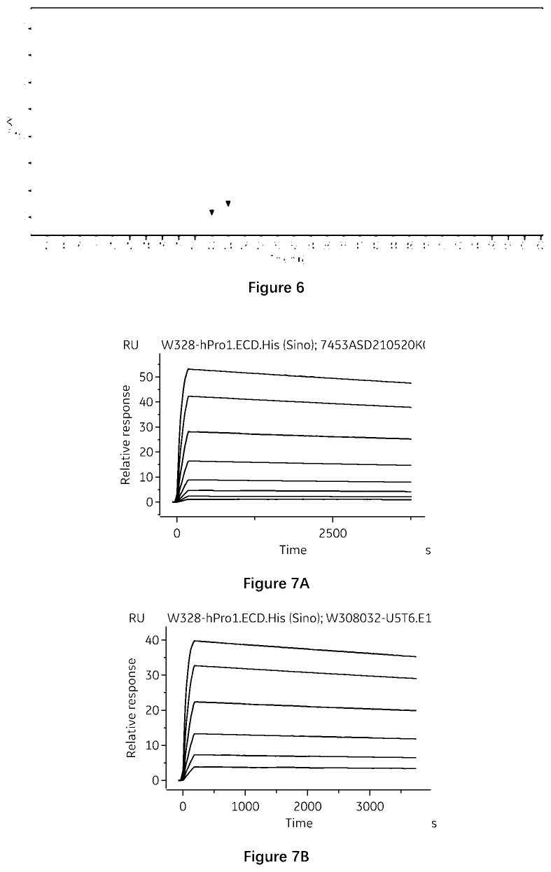

The determined retention time was 24.74 min, indicating W308032-U5T6.E17-57.uIgG1 has normal hydrophobicity (FIG. 6).

TABLE 6
Result of HIC-HPLC characterization

| | Retention | |
|---|---|---|
| Antibody Name | Time (min) | Comments |
| W308032-U5T6.E17-57.uIgG1 | 24.74 | Normal |

### 3.5 Surface Plasmon Resonance (SPR)

Binding affinity to human HER2 was detected using Biacore 8K. Testing antibody was captured on a CM5 sensor chip pre-immobilized with anti-human IgG Fc antibody. Human HER2 protein at different concentrations was injected over the sensor chip at a flow rate of 30 μL/min for an association phase of 180 s, followed by 3600 s dissociation. The chip was regenerated by 10 mM glycine (pH 1.5) after each binding cycle. Sensorgrams of blank surface and buffer channel were subtracted from the test sensorgrams. The experimental data was fitted by 1:1 binding model using Langmuir analysis. Molecular weight of 71.0 kDa was used to calculate the molar concentration of human HER2 protein as analyte.

Binding affinity to human CD47 was detected using Biacore 8K. Human CD47 protein was captured on a CM5 sensor chip pre-immobilized with streptavidin. Testing antibody at different concentrations was injected over the sensor chip at a flow rate of 30 μL/min for an association phase of 240 s, followed by 600-3600 s dissociation. The chip was regenerated by 10 mM glycine (pH 1.5) after each binding cycle. Sensorgrams of blank surface and buffer channel were subtracted from the test sensorgrams. The experimental data was fitted by 1:1 binding model using Langmuir analysis. Molecular weight of 147 kDa was used to calculate the molar concentration of testing antibody as analyte.

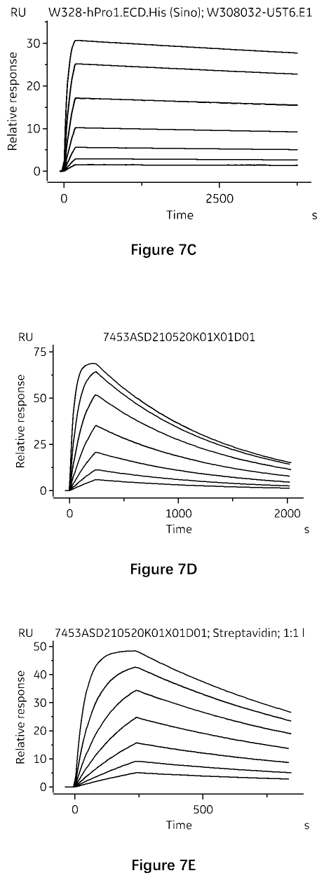

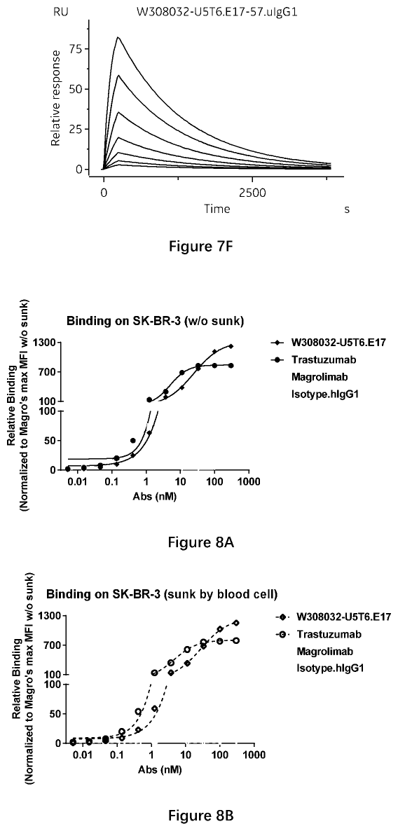

Results indicated W308032-U5T6.E17-57.uIgG1 maintained high affinity to human Her2 and relatively lower affinity to human CD47, the affinity of W308032-U5T6.E17-57.uIgG1 to HER2 is ˜50 folds higher compared to that of CD47 (FIGS. 7A-7F). FIGS. 7A-7C show 3 independent SPR results of the binding to HER2 and FIGS. 7D-7F show 3 independent SPR results of the binding to CD47.

3.6 Binding on Tumor Cells after Antigen Sink

Serial diluted antibodies were pre-incubated with human whole blood (1:10 dilution) or Jurkat cells (1.2˜1.8×10⁶ cells/well) at 4° C. for 1 hr followed by centrifugation and transfer of pre-sunk supernatant to a new plate. CFSE-labeled human tumor cells (0.8×10⁵ cells/well) were seeded into 96-well U-bottom plates and centrifuged at 1500 rpm at 4° C. for 4 min before removing the supernatant. Then pre-sunk supernatant was added to re-suspend cells and incubated at 4° C. for 1 hr followed by washing twice with 180 μL 1% BSA-PBS. The secondary antibody, Alexa647-conjugated goat anti-human IgG Fc, was added to re-suspend cells and incubated at 4° C. in dark for 0.5 hr followed by washing twice with 180 μL 1% BSA-PBS. Mean fluorescence intensity (MFI) was measured by FACS (BD Canto II) and analyzed by FlowJo Version software. Tumor cells were gated by CFSE and the binding EC₅₀ on tumor cells were calculated by using GraphPad Prism software: Nonlinear regression (curve fit)—log (agonist) vs. response—Variable slope.

After being sunk by human blood, W308032-U5T6.E17-57.uIgG1 showed binding on CD47/Her2 double positive SK-BR-3 cells at EC₅₀ of ˜27.69 nM, which was not affected compared to the binding without a pre-sunk by human blood (EC₅₀=24.36 nM). However, binding of Magrolimab on SK-BR-3 (EC₅₀=0.36 nM) was dramatically affected after being sunk by human blood (EC₅₀=6.41 nM), with about 18-fold shift in EC₅₀ (FIGS. 8A-B).

TABLE 8
Summary of antibody binding to SK-BR-3 cells in the
absence or presence of human blood cells as a sink

| | SK-BR-3 (w/o sunk) | | SK-BR-3 (sunk) | |
|---|---|---|---|---|
| | | Max | | Max |
| | EC₅₀ | Relative | EC₅₀ | Relative |
| Abs | (nM) | Binding | (nM) | Binding |
| W308032-U5T6.E17-57.uIgG1 | 24.36 | 1222 | 27.69 | 1152 |
| Trastuzumab | 5.01 | 827 | 4.57 | 793 |
| Magrolimab | 0.36 | 100 | 6.41 | 98 |
| Isotype.hIgG1 | NA | 6 | NA | 4 |

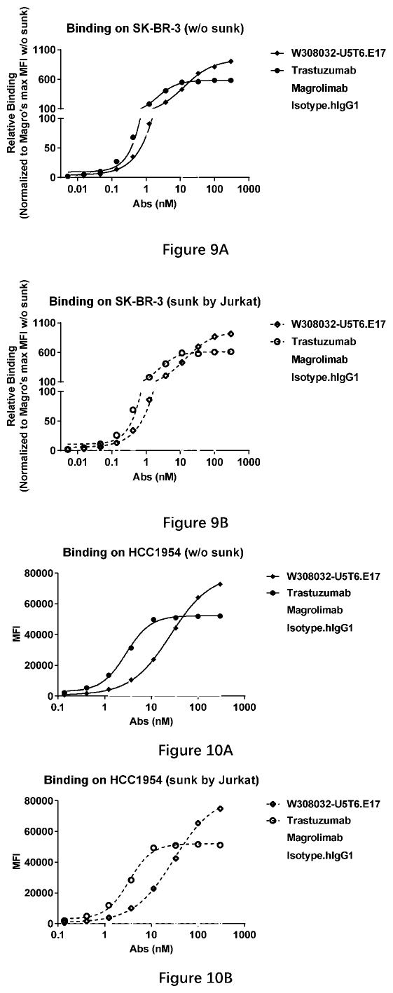

After being sunk by Jurkat cell, W308032-U5T6.E17-57.uIgG1 showed binding on SK-BR-3 cells at EC₅₀ of ˜13.29 nM, which was not affected compared to the binding without a pre-sunk by Jurkat cell (EC₅₀=12.55 nM). However, binding of Magrolimab on SK-BR-3 (EC₅₀=0.19 nM) was dramatically affected after being sunk by Jurkat cell (EC₅₀=1.53 nM), with about 8-fold shift in EC₅₀ (FIGS. 9A-B).

TABLE 9
Summary of antibody binding to SK-BR-3 cells in
the absence or presence of Jurkat cells as a sink

| | SK-BR-3 (w/o sunk) | | SK-BR-3 (sunk) | |
|---|---|---|---|---|
| | | Max | | Max |
| | EC₅₀ | Relative | EC₅₀ | Relative |
| Abs | (nM) | Binding | (nM) | Binding |
| W308032-U5T6.E17-57.uIgG1 | 12.55 | 905 | 13.29 | 913 |
| Trastuzumab | 2.11 | 582 | 2.25 | 613 |
| Magrolimab | 0.19 | 100 | 1.53 | 103 |
| Isotype.hIgG1 | NA | 9 | NA | 6 |

The antigen sink effect on the binding of antibody to CD47/Her2 double positive HCC1954 cell was also tested. After being sunk by Jurkat cell, W308032-U5T6.E17-57.uIgG1 showed binding on HCC1954 cells at EC₅₀ of ˜30.29 nM, which was not affected compared to the binding without a pre-sunk by Jurkat cell (EC₅₀=26.07 nM). However, binding of Magrolimab on HCC1954 (EC₅₀=0.44 nM) was affected after being sunk by Jurkat cell (EC₅₀=1.68 nM), with about 4-fold shift in EC₅₀ (FIGS. 10A-B).

TABLE 10
Summary of antibody binding to HCC1954 cells in
the absence or presence of Jurkat cells as a sink

| | HCC1954 (w/o sunk) | | HCC1954 (sunk) | |
|---|---|---|---|---|
| | EC₅₀ | Max | EC₅₀ | Max |
| Abs | (nM) | MFI | (nM) | MFI |
| W308032-U5T6.E17- | 26.07 | 72800 | 30.29 | 74800 |
| 57.uIgG1 | | | | |
| Trastuzumab | 2.86 | 52000 | 3.30 | 51700 |
| Magrolimab | 0.44 | 8504 | 1.68 | 8302 |
| Isotype.hIgG1 | NA | 192 | NA | 183 |

### 3.7 Binding on CD47 Single Positive Cell

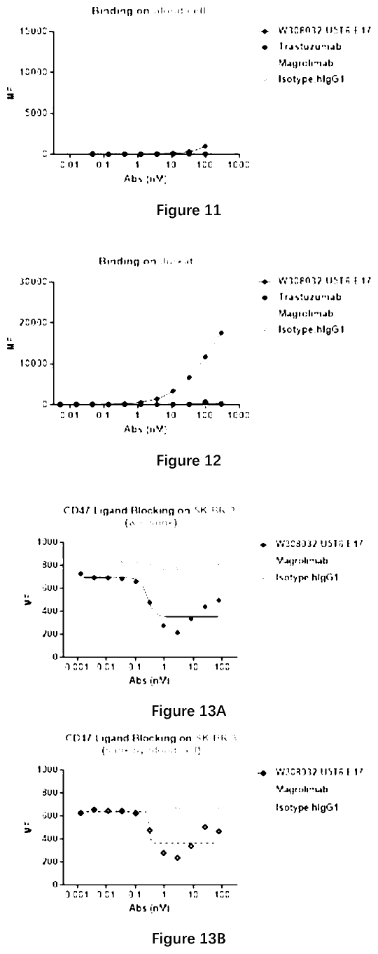

W308032-U5T6.E17-57.uIgG1 showed negligible binding on human blood cells (FIG. 11) and about 200-fold decreased binding on Jurkat cells compared to Magrolimab (FIG. 12).

TABLE 11
Summary of antibody binding to human blood cells

human blood cell

| | EC₅₀ | Max |
|---|---|---|
| Abs | (nM) | MFI |
| W308032-U5T6.E17-57.uIgG1 | >100 | 1001 |
| Trastuzumab | NA | 16 |
| Magrolimab | 0.28 | 11600 |
| Isotype.hIgG1 | NA | 30 |

TABLE 12
Summary of antibody binding to Jurkat cells

Jurkat

| | EC₅₀ | Max |
|---|---|---|
| Abs | (nM) | MFI |
| W308032-U5T6.E17-57.uIgG1 | >48 | 17600 |
| Trastuzumab | NA | 787 |
| Magrolimab | 0.76 | 25400 |
| Isotype.hIgG1 | NA | 302 |

3.8 CD47 Ligand Blocking on Tumor Cells after Antigen Sink

Serial diluted antibodies were pre-incubated with human whole blood (1:10 dilution) or Jurkat cells (1.2˜1.8×10⁶ cells/well) at 4° C. for 1 hr followed by centrifugation and transfer of pre-sunk supernatant to a new plate. CFSE-labeled human tumor cells (0.8×10⁵ cells/well) were seeded into 96-well U-bottom plates and centrifuged at 1500 rpm at 4° C. for 4 min before removing the supernatant. Then pre-sunk supernatant and mFc-tagged SIRPα (1 pg/mL) were added to re-suspend cells and incubated at 4° C. for 2 hrs followed by wash twice with 180 μL 1% BSA-PBS. The secondary antibody, Alexa647-conjugated goat anti-mouse IgG Fc was added to re-suspend cells and incubated at 4° C. in dark for 0.5 hr followed by wash twice with 180 μL 1% BSA-PBS. MFI was measured by FACS (BD Canto II) and analyzed by FlowJo Version software. Tumor cells were gated by CFSE and the inhibitory IC₅₀s on tumor cells were calculated by using GraphPad Prism software: Nonlinear regression (curve fit)—log (antagonist) vs. response—Variable slope.

After being sunk by human blood, W308032-U5T6.E17-57.uIgG1 blocked CD47 ligand binding on CD47/Her2 double positive SK-BR-3 cell with IC₅₀ of ˜0.30 nM, which was not affected compared to the blocking without a pre-sunk by human blood (IC₅₀=0.25 nM). However, CD47 ligand blocking of Magrolimab on SK-BR-3 (IC₅₀=0.06 nM) was dramatically affected after being sunk by human blood (IC₅₀=0.93 nM), with about 15-fold shift in IC₅₀ (FIGS. 13A-B).

TABLE 13
Summary of CD47 ligand blocking in the absence
or presence of human blood cells as a sink

| | SK-BR-3 (w/o sunk) | | SK-BR-3 (sunk) | |
|---|---|---|---|---|
| | IC₅₀ | Max | IC₅₀ | Max |
| Abs | (nM) | Inhibit | (nM) | Inhibit |
| W308032-U5T6.E17-57.uIgG1 | 0.25 | 88.1% | 0.30 | 81.8% |
| Magrolimab | 0.06 | 91.8% | 0.93 | 93.4% |
| Isotype.hIgG1 | NA | −6.5% | NA | 1.5% |

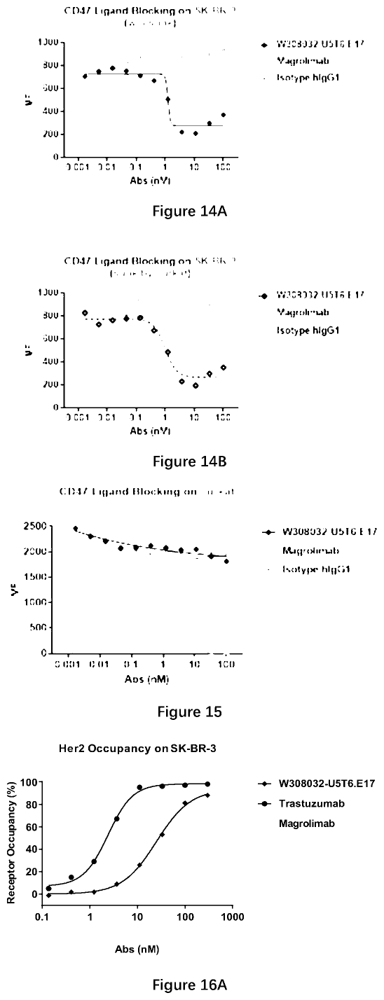

After being sunk by Jurkat cell, W308032-U5T6.E17-57.uIgG1 blocked CD47 ligand binding on CD47/Her2 double positive SK-BR-3 cell with IC₅₀ of ˜0.98 nM, which was not affected compared to the blocking without a pre-sunk by Jurkat cell (IC₅₀=1.24 nM). However, CD47 ligand blocking of Magrolimab on SK-BR-3 (IC₅₀=0.17 nM) was affected after being sunk by Jurkat cell (IC₅₀=0.44 nM), with about 3-fold shift in IC₅₀ (FIGS. 14A-B).

TABLE 14
Summary of CD47 ligand blocking in the absence
or presence of Jurkat cells as a sink

| | SK-BR-3 (w/o sunk) | | SK-BR-3 (sunk) | |
|---|---|---|---|---|
| | IC₅₀ | Max | IC₅₀ | Max |
| Abs | (nM) | Inhibit | (nM) | Inhibit |
| W308032-U5T6.E17-57.uIgG1 | 1.24 | 89.7% | 0.98 | 92.5% |
| Magrolimab | 0.17 | 97.3% | 0.44 | 98.5% |
| Isotype.hIgG1 | NA | 9.0% | NA | 1.6% |

In addition, W308032-U5T6.E17-57.uIgG1 showed almost no CD47 ligand blocking effect on Jurkat cell compared to Magrolimab (FIG. 15).

TABLE 15
Summary of CD47 ligand blocking effect on Jurkat cells

Jurkat

| | IC₅₀ | Max |
|---|---|---|
| Abs | (nM) | Inhibit |
| W308032-U5T6.E17-57.uIgG1 | NA | 14.8% |
| Magrolimab | 0.58 | 99.3% |
| Isotype.hIgG1 | NA | 13.7% |

### 3.9 Receptor Occupancy on Tumor Cells

Tumor cells were seeded into 96-well U-bottom plates and centrifuged at 1500 rpm at 4° C. for 4 min before removing the supernatant. Then bispecific antibodies with serial dilution were added to re-suspend cells and incubated at 4° C. for 1 hr followed by wash twice with 180 μL 1% BSA-PBS. The detection antibodies, biotin-labeled parental antibody at a saturating concentration, were added to re-suspend cells and incubated at 4° C. for 1 hr followed by wash twice with 180 μL 1% BSA-PBS. The secondary antibody Streptavidin PE was added to re-suspend cells and incubated at 4° C. in dark for 0.5 hr followed by washing twice with 180 μL 1% BSA-PBS. MFI was measured by FACS (BD Canto II) and analyzed by FlowJo Version software. Receptor occupancy (%) was calculated as: 100−(MFIₛₐₘₚₗₑ−MFI_{neg})/(MFI_{saturating}−MFI_{neg})×100. The EC₅₀ were calculated by using GraphPad Prism software: Nonlinear regression (curve fit)—log (agonist) vs. response—Variable slope.

With the help of Her2 binding arm, W308032-U5T6.E17-57.uIgG1 showed over 30% higher max CD47 occupancy than Magrolimab on CD47/Her2 double positive SK-BR-3 cell (FIGS. 16A-B).

TABLE 16
Summary of Her2 and CD47 occupancy on SK-BR-3 cell

| | SK-BR-3 (Her2 RO) | | SK-BR-3 (CD47 RO) | |
|---|---|---|---|---|
| | EC₅₀ | Max | EC₅₀ | Max |
| Abs | (nM) | RO % | (nM) | RO % |
| W308032-U5T6.E17- | 25.23 | 87.7 | 1.73 | 86.1 |
| 57.uIgG1 | | | | |
| Trastuzumab | 2.42 | 97.9 | NA | 4.1 |
| Magrolimab | NA | 0.0 | 0.35 | 65.1 |

### 3.10 ADCP Against Tumor Cells

Monocytes were isolated from PBMC by using human CD14 Microbeads and then differentiated to macrophages by treatment with rhM-CSF (50-100 ng/mL) for 6-8 days. Briefly, 30 μL of CFSE-labeled tumor cells, 50 μL of macrophages and 20 μL of serial diluted antibodies were seeded into 96-well U-bottom ultra-low plates. Phagocytosis of tumor cells was allowed at 37° C. for 2-3 hours. Cells were then stained with APC-conjugated CD14 antibody at 4° C. for 45 min and washed by 1% BSA-PBS before detected by FACS (BD Canto II) and analyzed by FlowJo Version software. Phagocytic activity was calculated as: index %=Percentage_{CFSE+/CD14-APC+}/(Percentage_{CFSE+/CD14-APC+}+ Percentage_{CFSE-/CD14-APC+})×100%. The phagocytic EC₅₀ were calculated by using GraphPad Prism software: Nonlinear regression (curve fit)—log (agonist) vs response—Variable slope.

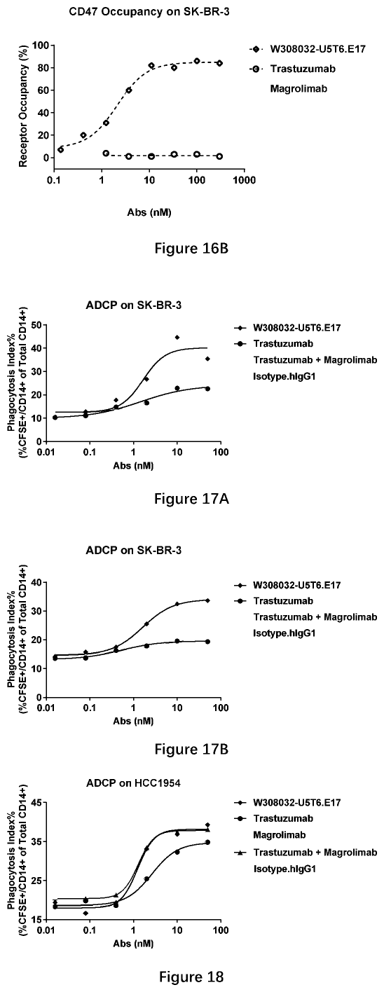

As shown in FIGS. 17A-B, W308032-U5T6.E17-57.uIgG1 showed increased ADCP efficacy compared to the combination of Trastuzuamb and Magrolimab on CD47/Her2 double positive SK-BR-3 cells in two independent experiments.

TABLE 17A
Summary of antibodies on SK-BR-3 cell (study 1)

SK-BR-3

| | EC₅₀ | Max |
|---|---|---|
| Abs | (nM) | Index % |
| W308032-U5T6.E17-57.uIgG1 | 1.33 | 44.6 |
| Trastuzumab | 1.44 | 22.8 |
| Trastuzumab + Magrolimab | 4.80 | 19.8 |
| Isotype.hIgG1 | NA | 10.2 |

TABLE 17B
Summary of antibodies on SK-BR-3 cell (study 2)

SK-BR-3

| | EC₅₀ | Max |
|---|---|---|
| Abs | (nM) | Index % |
| W308032-U5T6.E17-57.uIgG1 | 1.63 | 33.7 |
| Trastuzumab | 0.52 | 19.6 |
| Trastuzumab + Magrolimab | ~2.69 | 21.4 |
| Isotype.hIgG1 | NA | 10.8 |

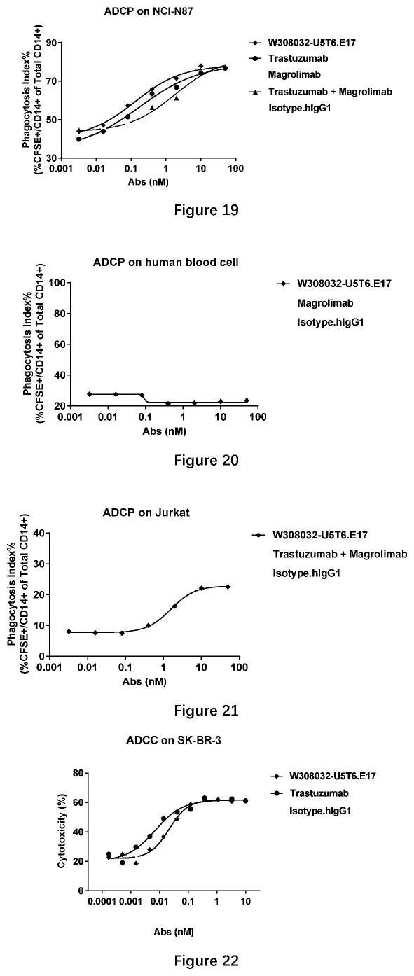

As shown in FIG. 18, W308032-U5T6.E17-57.uIgG1 showed comparable ADCP efficacy (EC₅₀=1.25 nM, max phagocytic index=39.3%) to the combination of Trastuzuamb and Magrolimab (EC₅₀=1.28 nM, max phagocytic index=38.1%) on CD47/Her2 double positive HCC1954 cell. As shown in FIG. 19, W308032-U5T6.E17-57.uIgG1 showed comparable ADCP efficacy (EC₅₀=0.13 nM, max phagocytic index=77.8%) to the combination of Trastuzuamb and Magrolimab (EC₅₀=1.77 nM, max phagocytic index=77.5%) on CD47/Her2 double positive NCI-N87 cell.

TABLE 18
Summary of antibodies on HCC1954

HCC1954

| | EC₅₀ | Max |
|---|---|---|
| Abs | (nM) | Index % |
| W308032-U5T6.E17-57.uIgG1 | 1.25 | 39.3 |
| Trastuzumab | 2.65 | 34.8 |
| Magrolimab | NA | 21.4 |
| Trastuzumab + Magrolimab | 1.28 | 38.1 |
| Isotype.hIgG1 | NA | 21.6 |

TABLE 19
Summary of antibodies on NCI-N87

NCI-N87

| | EC₅₀ | Max |
|---|---|---|
| Abs | (nM) | Index % |
| W308032-U5T6.E17-57.uIgG1 | 0.13 | 77.8 |
| Trastuzumab | 0.17 | 76.7 |
| Magrolimab | NA | 53.7 |
| Trastuzumab + Magrolimab | 1.77 | 77.5 |
| Isotype.hIgG1 | NA | 48.5 |

### ADCP Against CD47 Single Positive Cells

W308032-U5T6.E17-57.uIgG1 showed almost no ADCP efficacy on human blood cells (FIG. 20) and much less ADCP activity than Magrolimab on Jurkat cells (FIG. 21).

TABLE 20
Summary of antibodies on human blood cell

human blood cell

| | EC₅₀ | Max |
|---|---|---|
| Abs | (nM) | Index % |
| W308032-U5T6.E17-57.uIgG1 | NA | 27.6 |
| Magrolimab | 0.04 | 89.6 |
| Isotype.hIgG1 | NA | 31.1 |

TABLE 21
Summary of antibodies on Jurkat cell

Jurkat

| | EC₅₀ | Max |
|---|---|---|
| Abs | (nM) | Index % |
| W308032-U5T6.E17-57.uIgG1 | 1.57 | 22.5 |
| Trastuzumab + Magrolimab | 0.03 | 31.3 |
| Isotype.hIgG1 | NA | 6.7 |

### 3.11 ADCC Against Tumor Cells

NK cells were isolated from PBMC by using CD56 Positive Selection Kit. Briefly, 40 μL of tumor cells, 40 μL of NK cells and 20 μL of serial diluted antibodies were seeded into 96-well U-bottom plates. After incubation at 37° C. for 4 hr, cell mixtures were centrifuged at 1500 rpm for 5 min and 75 μL of supernatant were transferred for detection. Cell death was measured and calculated by using LDH Cytotoxicity Detection Kit (Roche) according to manufacturer's instructions. The cytotoxic EC₅₀ were calculated by using GraphPad Prism software: Nonlinear regression (curve fit)—log (agonist) vs response—Variable slope.

As shown in FIG. 22, W308032-U5T6.E17-57.uIgG1 showed potent ADCC efficacy (EC₅₀=21.42 pM, max cytotoxicity=61.9%) on CD47/Her2 double positive SK-BR-3 cells.

TABLE 22
Summary of ADCC on SK-BR-3

SK-BR-3

| | EC₅₀ | Max |
|---|---|---|
| Abs | (pM) | Cyto % |
| W308032-U5T6.E17-57.uIgG1 | 21.42 | 61.9 |
| Trastuzumab | 6.11 | 63.0 |
| Isotype.hIgG1 | NA | 27.5 |

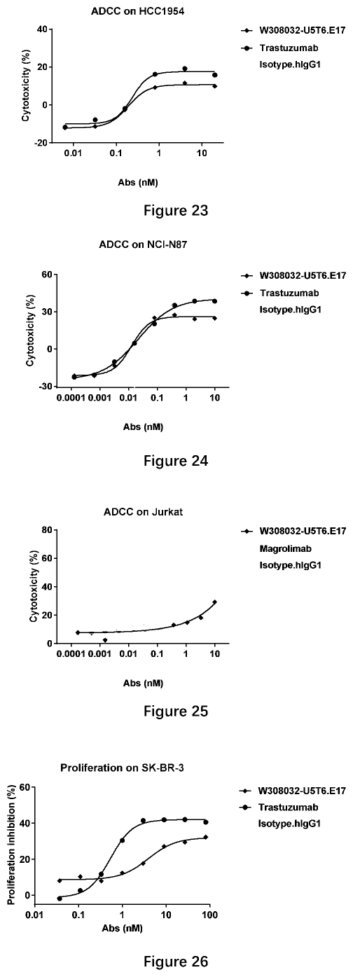

W308032-U5T6.E17-57.uIgG1 also showed potent ADCC efficacy on CD47/Her2 double positive HCC1954 cells (FIG. 23) and NCI-N87 cells (FIG. 24).

TABLE 23
Summary of ADCC on HCC1954

HCC1954

| | EC₅₀ | Max |
|---|---|---|
| Abs | (nM) | Cyto % |
| W308032-U5T6.E17-57.uIgG1 | 0.19 | 11.5 |
| Trastuzumab | 0.23 | 19.2 |
| Isotype.hIgG1 | NA | −8.2 |

TABLE 24
Summary of ADCC on NCI-N87

NCI-N87

| | EC₅₀ | Max |
|---|---|---|
| Abs | (pM) | Cyto % |
| W308032-U5T6.E17-57.uIgG1 | 12.62 | 27.4 |
| Trastuzumab | 21.63 | 38.6 |
| Isotype.hIgG1 | NA | −18.4 |

### ADCC Against CD47 Single Positive Cell

W308032-U5T6.E17-57.uIgG1 showed very weak ADCC efficacy on Jurkat cells only at high concentrations (FIG. 25).

TABLE 25
Summary of ADCC on Jurkat cell

Jurkat

| | EC₅₀ | Max |
|---|---|---|
| Abs | (pM) | Cyto % |
| W308032-U5T6.E17-57.uIgG1 | 753.1 | 29.2 |
| Magrolimab | NA | 13.7 |
| Isotype.hIgG1 | NA | 9.9 |

### 3.12 Inhibitory Effect on Proliferation of Tumor Cells

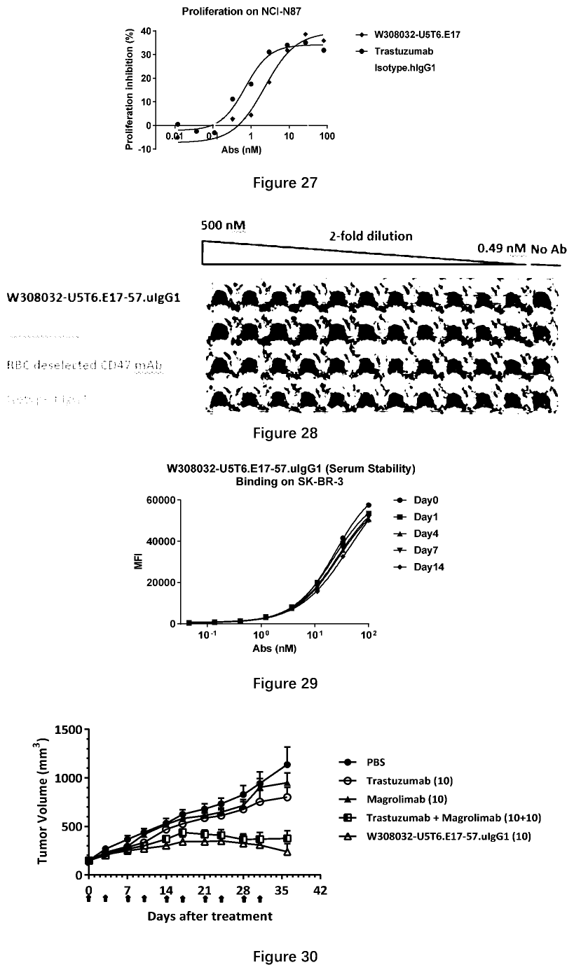

Briefly, 80 μL of tumor cells were seeded into 96-well black-wall plates. The next day, 20 μL of serial diluted antibodies were added into cells and incubated at 37° C. for 5-6 days. Then 50 μL/well CTG solution was added into cells and incubated at room temperature (RT) for 10 min followed by detection using EnVision. The inhibitory IC₅₀s were calculated by using GraphPad Prism software: Nonlinear regression (curve fit)—log (antagonist) vs response—Variable slope. W308032-U5T6.E17-57.uIgG1 showed a potent inhibitory effect on the proliferation of CD47/Her2 double positive SK-BR-3 cells (FIG. 26) and NCI-N87 cell (FIG. 27).

TABLE 26
Summary of inhibitory effect on SK-BR-3

SK-BR-3

| | IC₅₀ | Max |
|---|---|---|
| Abs | (nM) | Inhib % |
| W308032-U5T6.E17-57.uIgG1 | 3.94 | 32.3 |
| Trastuzumab | 0.54 | 42.0 |
| Isotype.hIgG1 | NA | 3.4 |

TABLE 27
Summary of inhibitory effect on NCI-N87

NCI-N87

| | IC₅₀ | Max |
|---|---|---|
| Abs | (nM) | Inhib % |
| W308032-U5T6.E17-57.uIgG1 | 2.28 | 38.6 |
| Trastuzumab | 0.70 | 35.1 |
| Isotype.hIgG1 | NA | 7.6 |

### 3.13 Hemagglutination

Anticoagulant fresh human whole blood was centrifuged at 2000 rpm for 10 min, and the plasma was discarded. Human blood cells were rinsed twice with DPBS. Briefly, 25 μL of 1:25 diluted human blood cells (i.e., the cells were resuspended using 25× the original blood volume) and 25 μL of serial diluted antibodies were added to U-bottom 96-well plate and incubated at 37° C. for 2 hr followed by taking pictures for detection.

W308032-U5T6.E17-57.uIgG1 was HA negative on human blood cells, while Magrolimab was HA positive on human blood cells (FIG. 28).

### 3.14 Serum Stability

Fresh human whole blood was statically incubated in tubes without anticoagulant for at least 30 min at room temperature (RT). Serum was collected after centrifuging the blood at 4000 rpm for 10 min. Antibodies were gently mixed with serum and incubated at 37° C. The one aliquot of serum-treated samples was collected and snap-frozen by liquid nitrogen on Day 0, Day 1, Day 4, Day 7, Day 14, respectively; and stored at −80° C. until ready for analysis. The samples were assessed their binding ability to tumor cells to reflect their stability. Briefly, tumor cells were seeded into 96-well U-bottom plates and centrifuged at 1500 rpm at 4° C. for 4 min before removing the supernatant. Then testing samples at various concentrations were added to re-suspend cells and incubated at 4° C. for 1 hr followed by wash twice with 180 μL 1% BSA-PBS. The secondary antibody, Alexa647-conjugated goat anti-human IgG Fc was added to re-suspend cells and incubated at 4° C. in dark for 0.5 hr followed by wash twice with 180 μL 1% BSA-PBS. MFI was measured by FACS (BD Canto II) and analyzed by FlowJo Version software. The binding EC₅₀s were calculated by using GraphPad Prism software: Nonlinear regression (curve fit)—log (agonist) vs. response—Variable slope.

W308032-U5T6.E17-57.uIgG1 was stable in human serum for at least 7 days and showed no decrease in binding on CD47/Her2 double positive SK-BR-3 cell. When incubated with human serum for 14 days, W308032-U5T6.E17-57.uIgG1 showed a slight (less than 2-fold) decrease in binding potency on CD47/Her2 double positive SK-BR-3 cells (FIG. 29).

TABLE 28
Summary of serum stability result

SK-BR-3

| | EC₅₀ | Max |
|---|---|---|
| Abs | (nM) | MFI |
| UST6.E17 (Day 0) | 23.75 | 57500 |
| U5T6.E17 (Day 1) | 22.82 | 53500 |
| UST6.E17 (Day 4) | 27.06 | 50600 |
| UST6.E17 (Day 7) | 26.65 | 51600 |
| UST6.E17 (Day 14) | 42.55 | 50100 |

### 4.1 Rodent Efficacy

### 4.1.1 HCC1954 Xenograft Model

Female CB-17 SCID mice (Beijing Vital River Laboratory Animal Technology Co., Ltd) of 6-8 week-old were used in the study. HCC1954 cells were maintained in vitro as a monolayer culture in RPMI-1640 medium supplemented with 10% fetal bovine serum at 37° C. in an atmosphere of 5% CO₂ in air. For the xenograft model, HCC1954 tumor cells in an exponential growth phase were harvested and each mouse was inoculated subcutaneously at the right forearm armpit with HCC1954 tumor cells (3.0×10⁶ cells/0.2 mL, 1:1 DPBS and Matrigel (Corning)). When the average tumor volume reached ˜147 mm³, animals were randomly grouped into 12 groups, and received the first dose of antibody treatment (Day 0), followed with twice a week treatment intraperitoneally for a total of 10 injections. Mice were weighed, and tumor growth was measured twice a week using calipers. Tumor volume was calculated with the formula (½ (length×width²). The results were represented by mean and the standard error (Mean±SEM). Data were analyzed using Two way ANOVA Bonferroni posttests with Prism and p<0.05 was considered to be statistically significant. All the procedures related to animal handling care and the treatment in the study were performed according to the guidelines approved by the Institutional Animal Care and Use Committee (IACUC) of WuXi Biologics following the guidance of the Association for Assessment and Accreditation of Laboratory Animal Care (AAALAC). The details of grouping and treatment for HCC1954 xenograft model were shown in Table 29.

TABLE 29
Grouping and Treatment for HCC1954 xenograft model

| | | Numbers/ | Dosage | Dosing | Dosing | Dosing |
|---|---|---|---|---|---|---|
| Groups | Treatment | group | (mg/kg) | Route | Frequency | times |
| G1 | PBS | 8 | / | IP | BIW | 10 times |
| G2 | Trastuzumab | 8 | 10 | IP | BIW | 10 times |
| G3 | Magrolimab | 8 | 10 | IP | BIW | 10 times |
| G4 | Pertuzumab | 8 | 10 | IP | BIW | 10 times |
| G5 | Trastuzumab + Magrolimab | 8 | 10 + 10 | IP | BIW | 10 times |
| G6 | Trastuzumab + Pertuzumab | 8 | 10 + 10 | IP | BIW | 10 times |
| G7 | Trastuzumab + Magrolimab + | 8 | 10 + 10 + 10 | IP | BIW | 10 times |
| | Pertuzumab | | | | | |
| G8 | W308032-U5T6.E17-57.uIgG1 | 8 | 30 | IP | BIW | 10 times |
| G9 | W308032-U5T6.E17-57.uIgG1 | 8 | 10 | IP | BIW | 10 times |
| G10 | W308032-U5T6.E17-57.uIgG1 | 8 | 3 | IP | BIW | 10 times |
| G11 | W308032-U5T6.E17-57.uIgG1 | 8 | 1 | IP | BIW | 10 times |
| G12 | W308032-U5T6.E17- | 8 | 10 + 10 | IP | BIW | 10 times |
| | 57.uIgG1 + Pertuzumab | | | | | |

Except for two mice (one from W308032-U5T6.E17-57.uIgG1 3 mg/kg group and one from W308032-U5T6.E17-57.uIgG1 1 mg/kg group) that were found dead on Day 28 and Day 17, and one mouse from Pertuzumab group that was euthanized due to tumor volume reached 2500 mm³, all the other mice appeared normal during the experiment and slowly gained weight, which indicated the antibodies were not toxic (data not shown).

As shown in FIG. 30 and summarized in Table 30, compared with the PBS group, Trastuzumab+Magrolimab (Day 36, TGI 76.8%) could significantly inhibit tumor growth, and had a significant difference from Day 7; while the Trastuzumab (Day 36, TGI 33.6%) and Magrolimab (Day 36, TGI 18.5%) mono-therapy groups showed a slight inhibition on tumor growth, indicating the synergistic effect of CD47 and Her2. W308032-U5T6.E17-57.uIgG1 (10 mg/kg) showed a slightly better efficacy than the Trastuzumab+Magrolimab combination, and a significantly better efficacy than Trastuzumab or Magrolimab mono-therapy (FIG. 30).

TABLE 30
Summary of TGI and P value on Day 36 in HCC1954 xenograft model

| | | Tumor Volume (mm³) | | | TGI (%) |
|---|---|---|---|---|---|
| | | Mean ± SEM | | P | Day 36 |
| | | Before | Day 36 post | value | post first |
| Group | Antibody Name | treatment | first treatment | (G1) | treatment |
| G1 | PBS | 148.15 ± 8.13 | 1135.72 ± 183.05 | — | — |
| G2 | Trastuzumab | 145.90 ± 6.38 | 801.97 ± 102.40 | ** | 33.6 |
| G3 | Magrolimab | 144.74 ± 6.06 | 949.72 ± 102.46 | — | 18.5 |
| G4 | Pertuzumab | 145.22 ± 6.62 | 1046.32 ± 137.80 | — | 8.76 |
| G5 | Trastuzumab + | 147.70 ± 8.74 | 376.88 ± 81.39 | ** | 76.8 |
| | Magrolimab | | | | |
| G6 | Trastuzumab + | 145.25 ± 5.39 | 632.45 ± 95.83 | ** | 50.7 |
| | Pertuzumab | | | | |
| G7 | Trastuzumab + | 146.02 ± 7.73 | 95.18 ± 8.25 | ** | 105.2 |
| | Magrolimab + | | | | |
| | Pertuzumab | | | | |
| G8 | W308032-U5T6.E17- | 148.04 ± 8.00 | 146.95 ± 39.52 | ** | 100.1 |
| | 57.uIgG1(30) | | | | |
| G9 | W308032-U5T6.E17- | 145.96 ± 5.46 | 237.50 ± 85.27 | ** | 90.7 |
| | 57.uIgG1(10) | | | | |
| G10 | W308032-U5T6.E17- | 145.28 ± 5.62 | 385.85 ± 86.30 | ** | 75.6 |
| | 57.uIgG1(3) | | | | |
| G11 | W308032-U5T6.E17- | 146.88 ± 5.75 | 772.05 ± 85.93 | ** | 36.7 |
| | 57.uIgG1(1) | | | | |
| G12 | W308032-U5T6.E17- | 148.39 ± 7.88 | 89.30 ± 19.29 | ** | 106.0 |
| | 57.uIgG1(10) + | | | | |
| | Pertuzumab | | | | |

Note:
(Two way ANOVA Bonferroni posttests, *p < 0.05; **p < 0.01)

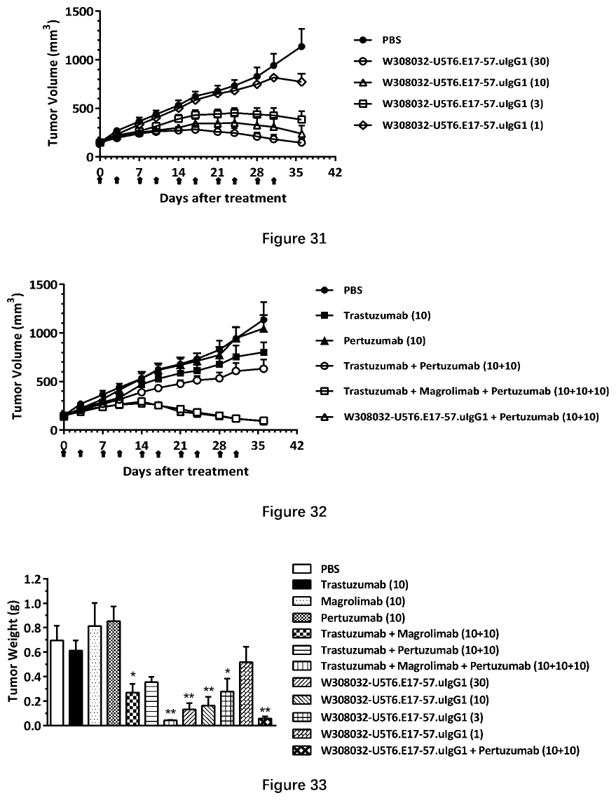

W308032-U5T6.E17-57.uIgG1 (30 mg/kg, 10 mg/kg, 3 mg/kg, 1 mg/kg) could inhibit tumor growth (Day 36, TGI 100.1%, 90.7%, 75.6%, 36.7%, respectively), and had a significant difference from Day 3 (30 mg/kg), Day 7 (10 mg/kg), Day 10 (3 mg/kg), respectively, indicating that W308032-U5T6.E17-57.uIgG1 had dose-dependent anti-tumor efficacy (FIG. 31).

The combinatory therapy of trastuzumab with pertuzumab has been approved for clinical usage and shown improved anti-tumor efficacy, as compared to trastuzumab monotherapy. To evaluate whether W308032-U5T6.E17-57.uIgG1 also has the potential to be used with pertuzumab to further increase the anti-tumor activity, the anti-tumor activity of the combination therapy was also examined in the HCC1954 xenograft model. As shown in FIG. 32 and summarized in Table 30, compared with the PBS group, Trastuzumab+Pertuzumab (Day 36, TGI 50.7%) and Trastuzumab+Magrolimab+Pertuzumab (Day 36, TGI 105.2%) combination groups could significantly inhibit tumor growth, and had significant difference from Day 7; while Trastuzumab (Day 36, TGI 33.6%) and Pertuzumab (Day 36, TGI 8.76%) monotherapy groups showed only slight inhibition on tumor growth. W308032-U5T6.E17-57.uIgG1+Pertuzumab combo group (Day 36, TGI 106.0%) showed comparable anti-tumor efficacy to Trastuzumab+Magrolimab+Pertuzumab combo group (Day 36, TGI 105.2%).

All above conclusions were also confirmed by the tumor weight data obtained on Day 36 post first treatment (FIG. 33). Data were shown as Mean+SEM.

### 4.1.2 NCI-N87 Xenograft Model

Female CB-17 SCID mice (Beijing Vital River Laboratories Research Models and Services) of 4-8 week-old were used in the study. NCI-N87 cells were maintained in vitro as a monolayer culture in RPMI-1640 medium supplemented with 10% fetal bovine serum at 37° C. in an atmosphere of 5% CO₂ in the air. For the xenograft model, NCI-N87 tumor cells in an exponential growth phase were harvested and each mouse was inoculated subcutaneously in the right front flank region with NCI-N87 tumor cells (1×10⁷ cells/0.1 mL, 1:1 PBS and Matrigel). When the average tumor volume reached ˜157 mm³, animals were randomly grouped into 10 groups, and received the first dose of treatment (Day 0), followed with twice a week treatment of antibodies intraperitoneally for a total of 11 injections, except once a week treatment of Enhertu intraperitoneally for a total of 6 injections. Mice were weighed, and tumor growth was measured twice a week using calipers. Tumor volume was calculated with the formula (% (length×width²).

All the results were represented by mean and the standard error (Mean±SEM). Data were analyzed using Two way ANOVA Bonferroni posttests or One way ANOVA Dunnett's multiple comparisons test in R-a language and environment for statistical computing and graphics (version 3.3.1) and p<0.05 was considered to be statistically significant. All the procedures related to the care and use of animals were approved by the Institutional Animal Care and Use Committee (IACUC) of CrownBio in accordance with the regulations of the Association for Assessment and Accreditation of Laboratory Animal Care (AAALAC). The details of grouping and treatment for NCI-N87 xenograft model were shown in Table 31.

TABLE 31
Grouping and Treatment for NCI-N87 xenograft model

| | | Number/ | Dosage | Dosing | Dosing | Dosing |
|---|---|---|---|---|---|---|
| Groups | Treatment | group | (mg/kg) | Route | Frequency | times |
| G1 | Vehicle control (PBS) | 8 | / | IP | BIW | 11 times |
| G2 | Herceptin (Trastuzumab) + Magrolimab | 8 | 10 + 10 | IP | BIW | 11 times |
| G3 | W308032-U5T6.E17-57.uIgG1 | 8 | 5 | IP | BIW | 11 times |
| G4 | W308032-U5T6.E17-57.uIgG1 | 8 | 10 | IP | BIW | 11 times |
| G5 | W308032-U5T6.E17-57.uIgG1 | 8 | 20 | IP | BIW | 11 times |
| G6 | W308032-U5T6.E17-57.uIgG1 | 8 | 40 | IP | BIW | 11 times |
| G7 | Enhertu | 8 | 10 | IP | QW | 6 times |
| G8 | Paclitaxel | 8 | 10 | IP | BIW | 11 times |
| G9 | W308032-U5T6.E17-57.uIgG1 + | 8 | 10 + 10 | IP | BIW | 11 times |
| | Paclitaxel | | | | | |
| G10 | W308032-U5T6.E17-57.uIgG1 + | 8 | 20 + 10 | IP | BIW | 11 times |
| | Paclitaxel | | | | | |

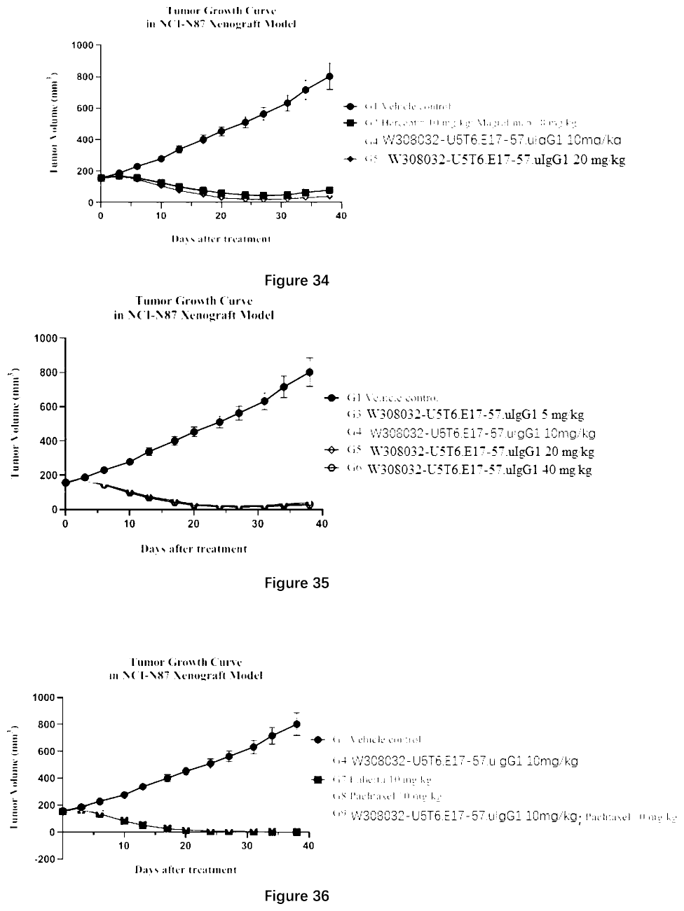

As shown in FIG. 34 and summarized in Table 32, compared with vehicle control group, Herceptin (Trastuzumab, 10 mg/kg)+Magrolimab (10 mg/kg) could significantly inhibit tumor growth (Day 38, TGI 111.92%), and W308032-U5T6.E17-57.uIgG1 (10 mg/kg & 20 mg/kg) showed comparable efficacy to Herceptin+Magrolimab combo therapy (Day 38, TGI 116.04% & 118.17%, respectively). As shown in FIG. 35, W308032-U5T6.E17-57.uIgG1 (5 mg/kg, 10 mg/kg, 20 mg/kg, 40 mg/kg) showed dose-dependent anti-tumor efficacy (Day 38, TGI 112.22%, 116.04%, 118.17%, 119.99%, respectively).

The combination of trastuzumab with chemotherapy has been approved for clinical usage and shown improved anti-tumor efficacy, as compared to trastuzumab monotherapy. To evaluate whether W308032-U5T6.E17-57.uIgG1 also has the potential to be used with chemotherapy to further increase the anti-tumor activity, the combo therapies' anti-tumor activity was also examined in the NCI-N87 xenograft model. As shown in FIG. 36 and summarized in Table 32, W308032-U5T6.E17-57.uIgG1 (10 mg/kg) in combination with paclitaxel (10 mg/kg) resulted in significant anti-tumor efficacy with TGI of 117.57%, which was comparable to the efficacy of Enhertu (10 mg/kg) with TGI of 124.19%.

TABLE 32
Summary of TGI and P value on Day 38 in NCI-N87 xenograft model

| | | Tumor Volume (mm³) | | TGI |
|---|---|---|---|---|
| | | Mean ± SEM | P | (%) |
| | | Day 38 post | value | Day 38 post |
| Group | Antibody Name | first treatment | (G1) | first treatment |
| G1 | Vehicle control | 802.17 ± 83.35 | — | — |
| G2 | Herceptin + | 79.88 ± 13.38 | 0.00073 | 111.92 |
| | Magrolimab | | | |
| G3 | W308032-U5T6.E17- | 77.99 ± 9.12 | 0.00131 | 112.22 |
| | 57.UIGG1 (5 mg/kg) | | | |
| G4 | W308032-U5T6.E17- | 53.38 ± 6.51 | <0.001 | 116.04 |
| | 57.UIGG1 (10 mg/kg) | | | |
| G5 | W308032-U5T6.E17- | 39.55 ± 3.73 | <0.001 | 118.17 |
| | 57.UIGG1 (20 mg/kg) | | | |
| G6 | W308032-U5T6.E17- | 27.85 ± 5.60 | <0.001 | 119.99 |
| | 57.UIGG1 (40 mg/kg) | | | |
| G7 | Enhertu | 0.78 ± 0.78 | <0.001 | 124.19 |
| G8 | Paclitaxel | 505.07 ± 43.52 | 0.955 | 46.04 |
| G9 | W308032-U5T6.E17- | 43.46 ± 6.58 | <0.001 | 117.57 |
| | 57.UIGG1 (10 mg/kg) + | | | |
| | Paclitaxel | | | |
| G10 | W308032-U5T6.E17- | 29.39 ± 5.76 | <0.001 | 119.74 |
| | 57.UIGG1 (20 mg/kg) + | | | |
| | Paclitaxel | | | |

### 4.1.3 JIMT-1 Xenograft Model

Efficacy in another trastuzumab-resistant breast cancer cell JIMT-1 xenograft model was also evaluated. Female CB-17 SCID mice (Shanghai Lingchang Bio-Technology. Co., Ltd) of 9-10 week-old were used in the study. JIMT-1 cells were maintained in vitro as a monolayer culture in DMEM medium supplemented with 10% fetal bovine serum at 37° C. in an atmosphere of 5% CO₂ in the air. For the xenograft model, JIMT-1 tumor cells in an exponential growth phase were harvested and each mouse was inoculated subcutaneously in the right upper flank region with JIMT-1 tumor cells (5×10⁶ cells/0.1 mL PBS). When the average tumor volume reached ˜134 mm³, animals were randomly grouped into 6 groups, and received the first dose of treatment (Day 0), followed with twice a week treatment of antibodies intraperitoneally for a total of 10 injections and another 4-week observation period after the last injection. Mice were weighed, and tumor growth was measured twice a week using calipers. Tumor volume was calculated with the formula (½ (length×width²).

All the results were represented by mean and the standard error (Mean±SEM). Data were analyzed using Two way ANOVA Bonferroni posttests or One way ANOVA Dunnett's multiple comparisons test with Prism and p<0.05 was considered to be statistically significant. All the procedures related to the care and use of animals were approved by the Institutional Animal Care and Use Committee (IACUC) of CrownBio in accordance with the regulations of the Association for Assessment and Accreditation of Laboratory Animal Care (AAALAC). The details of grouping and treatment for JIMT-1 xenograft model were shown in Table 33.

TABLE 33
Grouping and Treatment for JIMT-1 xenograft model

| | | Number/ | Dosage | Dosing | Dosing | Dosing |
|---|---|---|---|---|---|---|
| Groups | Treatment | group | (mg/kg) | Route | Frequency | times |
| G1 | Vehicle control (PBS) | 8 | / | IP | BIW | 10 times |
| G2 | Trastuzumab | 8 | 10 | IP | BIW | 10 times |
| G3 | Trastuzumab + Paclitaxel | 8 | 10 + 10 | IP | BIW | 10 times |
| G4 | W308032-U5T6.E17-57.uIgG1 | 8 | 10 | IP | BIW | 10 times |
| G5 | W308032-U5T6.E17-57.uIgG1 + | 8 | 10 + 10 | IP | BIW | 10 times |
| | Paclitaxel | | | | | |
| G6 | Enhertu | 8 | 10 | IP | BIW | 10 times |

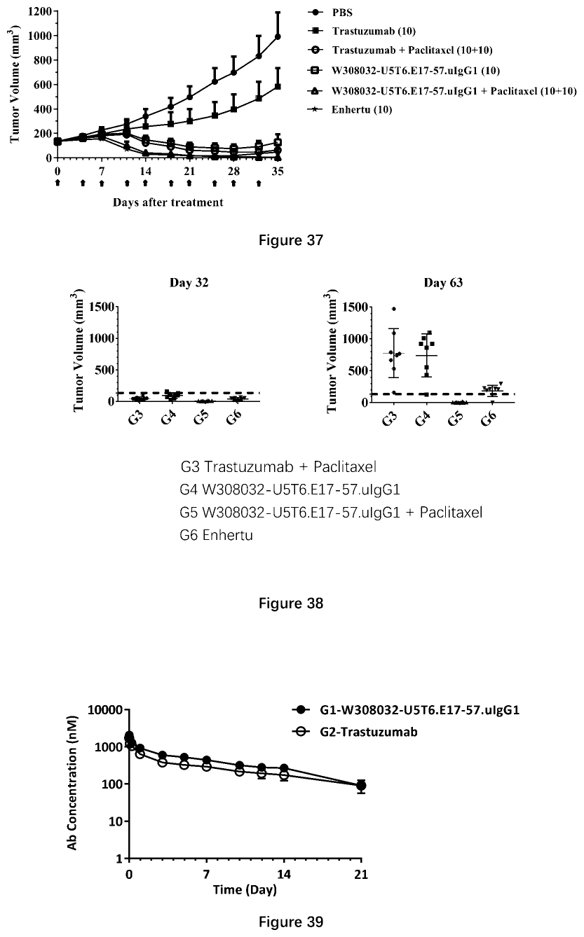

Animals were treated twice a week for total 10 doses in 5 weeks, followed by observation for 4 weeks in the absence of treatment. On Day 35 (three days after the last dosing), W308032-U5T6.E17-57.uIgG1 showed dose-dependent inhibition of tumor growth and comparable efficacy to combination of trastuzumab and magrolimab (data not shown). As shown in FIG. 37 and summarized in Table 34, W308032-U5T6.E17-57.uIgG1 in combination with paclitaxel showed better efficacy than bispecific antibody or trastuzumab mono treatment in tumor growth inhibition, which was comparable to the efficacy of Enhertu.

As shown in FIG. 38, on Day 63 (thirty-one days after the last dosing), W308032-U5T6.E17-57.uIgG1 in combination with paclitaxel showed almost no rebound in tumor volume, with tumor free in 4 out of 8 mice each group. However, Enhertu showed obvious rebound over initial tumor volume, with tumor free in only 1 out of 8 mice each group.

TABLE 34
Summary of TGI and P value on Day 35 in JIMT-1 xenograft model

| | | Tumor Volume (mm³) | | | P |
|---|---|---|---|---|---|
| | Test | Before | 35 days post | TGI | value |
| Group | articles | grouping | grouping | (%) | (G1) |
| G1 | PBS | 134.32 ± 3.98 | 991.99 ± 70.50 | — | — |
| G2 | Trastuzumab | 134.39 ± 3.92 | 582.01 ± 54.26 | 47.81 | <0.001 |
| G3 | Trastuzumab + | 134.35 ± 3.88 | 63.35 ± 15.95 | 108.28 | <0.001 |
| | Paclitaxel | | | | |
| G4 | W308032-U5T6.E17- | 134.19 ± 3.80 | 126.47 ± 23.43 | 100.90 | <0.001 |
| | 57.uIgG1 | | | | |
| G5 | W308032-U5T6.E17- | 134.17 ± 3.85 | 6.44 ± 1.18 | 114.89 | <0.001 |
| | 57.uIgG1 + | | | | |
| | Paclitaxel | | | | |
| G6 | Enhertu | 134.35 ± 3.92 | 46.05 ± 9.90 | 110.29 | <0.001 |

### 4.2 Rodent Pharmacokinetics

Female SD rat (Beijing Vital River Laboratory Animal Technology Co., Ltd) of 9-11 week-old were used in the study. Animals were housed in a specific pathogen free (SPF) barrier at Shanghai Model Organisms Center, Inc. with 2 animals per individual ventilated cage (IVC) and acclimated for one week after arrival. For pharmacokinetics (PK) study, each rat was given a single dose of antibody (15 mg/kg, IV bolus) on Day 0 and blood samples were taken from the eyes and transferred to the EDTA tubes. The tubes were centrifuged at 8000 rpm (6010 g) for 5 minutes at 4° C. and then the plasma was collected and stored at −20° C. All samples were uniquely identified to indicate origin and collection time. The schedule for sample collection per rat per dose was shown in Table 35.

TABLE 35
Sample collection schedule for rat PK study

| Matrix | Volume | Group | Time-points |
|---|---|---|---|
| Serum | 200 μL | G1: W308032-U5T6.E17- | Pre-dose, 0.5 h, 2 h, 6 h, 24 h, |
| | | 57.uIgG1 | Day 3 (72 h), Day 5 (120 h), |
| | | | Day 7 (168 h), Day 10 (240 h), |
| | | G2: Trastuzumab | Day 12 (288 h), Day 14 (336 h), |
| | | | Day 21 (504 h) post-dose |

The concentrations of analytes in serum were determined by using a bioanalytical ELISA method. Briefly, 96-well ELISA plates were coated overnight at 2-8° C. with recombinant human HER2/ErB2 protein (His tag) in carbonate-bicarbonate buffer as capturing antibody. After wash and blocking, serial diluted plasma samples were added and then biotin-labeled recombinant human CD47 protein (HER2+CD47 method for G1) or goat anti-human IgG-Biotin (HER2+Fc method for G2) was used as detection antibody. HRP-streptavidin and TMB substrate were used for color development. The reaction was stopped after approximate 5′˜10 minutes through the addition of 2M HCL. The absorbance was read at 450 nm and 540 nm using a microplate spectrophotometer (SpectraMax® M5e). The GD value of the samples were substituted into the standard curve to obtain the plasma antibody concentration. The serum concentration of antibody in rat was subjected to a non-compartmental pharmacokinetic analysis by using the Phoenix WinNonlin software (version 8. 1, Pharsight, Mountain View, CA). The linear/log trapezoidal rule was applied in obtaining the PK parameters. All the PK parameters were calculated without antibody concentration less than 1% of Cmax.

As shown in FIG. 39, W308032-U5T6.E17-57.uIgG1 showed comparable rodent PK (t½=153 h, Table 36) to Trastuzumab (t½=184 h, Table 37).

TABLE 36
Summary of pharmacokinetics parameters for W308032-U5T6.E17-57.uIgG1

| | PK parameters | | | | |
|---|---|---|---|---|---|
| | G1: W308032-U5T6.E17-57.uIgG1 | | | | |
| | Method | | | | |
| | HER2 + CD47 | | | | |
| | Animal ID | | | | |
| | #2712 | #2713 | #2714 | #2715 | Mean |
| t½ (h) | 138 | 189 | 146 | 139 | 153 |
| Cmax (nM) | 1891 | 2136 | 2068 | 2109 | 2051 |
| AUC_(0-t) (h*nM) | 195625 | 204671 | 204190 | 181370 | 196464 |
| Cl_obs (ml/h/kg) | 0.486 | 0.431 | 0.458 | 0.516 | 0.473 |
| MRTINF_obs (h) | 205 | 251 | 209 | 198 | 216 |
| Vss_obs (mL/kg) | 99.4 | 108 | 95.8 | 102 | 102 |

TABLE 37
Summary of pharmacokinetics parameters for Trastuzumab

| | PK parameters | | | | |
|---|---|---|---|---|---|
| | G2: Trastuzumab | | | | |
| | Method | | | | |
| | HER2 + Fc | | | | |
| | Animal ID | | | | |
| | #2717 | #2718 | #2719 | #2720 | Mean |
| t½ (h) | 225 | 140 | 118 | 255 | 184 |
| Cmax (nM) | 2048 | 1808 | 1493 | 1555 | 1726 |
| AUC_(0-t) (h*nM) | 183728 | 102471 | 92819 | 162739 | 135439 |
| Cl_obs (ml/h/kg) | 0.450 | 0.937 | 1.056 | 0.461 | 0.726 |
| MRTINF_obs (h) | 307 | 176 | 156 | 378 | 254 |
| Vss_obs (mL/kg) | 138 | 165 | 164 | 174 | 160 |

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

### REFERENCES

[1] Meike E W Logtenberg, Ferenc A Scheeren, Ton N Schumacher. The CD47-SIRPα Immune Checkpoint. Immunity. 2020 May 19; 52(5):742-752.
[2] E J Brown, W A Frazier. Integrin-associated protein (CD47) and its ligands. Trends Cell Biol. 2001 March; 11(3):130-5.
[3] Mark P Chao, Irving L Weissman, Ravindra Majeti. The CD47-SIRPα pathway in cancer immune evasion and potential therapeutic implications. Curr Opin Immunol. 2012 April; 24(2):225-32.
[4] I Rubin, Y Yarden. The basic biology of HER2. Ann Oncol. 2001; 12 Suppl 1:S3-8
[5] Li-Chung Tsao et.al. CD47 blockade augmentation of trastuzumab antitumor efficacy dependent on antibody-dependent cellular phagocytosis. JCI Insight. 2019 Dec. 19; 4(24):e131882.
[6] Tiia J Honkanen et.al. Prognostic and predictive role of spatially positioned tumour infiltrating lymphocytes in metastatic HER2 positive breast cancer treated with trastuzumab. Sci Rep. 2017 Dec. 21; 7(1):18027.

1. A polypeptide complex or antigen-binding portion thereof, comprising a first antigen-binding moiety that specifically binds to HER2 and a second antigen-binding moiety that specifically binds to CD47, wherein:
the first antigen-binding moiety comprises a first heavy chain variable domain (VH1) and a first light chain variable domain (VL1), and the VH1 comprises a heavy chain complementarity determining region (HCDR) 1 comprising the amino acid sequence of SEQ ID NO: 1, a HCDR2 comprising the amino acid sequence of SEQ ID NO: 2, a HCDR3 comprising the amino acid sequence of SEQ ID NO: 3, and the VL1 comprises a light chain complementarity determining region (LCDR) 1 comprising the amino acid sequence of SEQ ID NO: 4, a LCDR2 comprising the amino acid sequence of SEQ ID NO: 5, and a LCDR3 comprising the amino acid sequence of SEQ ID NO: 6; and
the second antigen-binding moiety comprises a second heavy chain variable domain (VH2) and a second light chain variable domain (VL2), and the VH2 comprises a HCDR1 comprising the amino acid sequence of SEQ ID NO: 7, a HCDR2 comprising the amino acid sequence of SEQ ID NO: 8, a HCDR3 comprising the amino acid sequence of SEQ ID NO: 9, and the VL2 comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

2. The polypeptide complex or antigen-binding portion thereof of claim 1, wherein the first antigen-binding moiety and the second antigen-binding moiety are independently selected from the following format: immunoglobin and antigen-binding portions thereof such as Fab, F(ab)′₂ or scFv.

3. The polypeptide complex or antigen-binding portion thereof of claim 1 or 2, wherein:
the first antigen-binding moiety comprises the VH1 operably linked to a first T cell receptor (TCR) constant region (C1), and the VL1 operably linked to a second TCR constant region (C2), the second antigen-binding moiety comprises the VH2 operably linked to an antibody heavy chain CH1 domain and the VL2 operably linked to an antibody light chain constant (CL) domain; or
the first antigen-binding moiety comprises the VH1 operably linked to an antibody heavy chain CH1 domain, and the VL1 operably linked to an antibody light chain constant (CL) domain, the second antigen-binding moiety comprises the VH2 operably linked to the C1 region and the VL2 operably linked to the C2 region;
wherein C1 and C2 are capable of forming one or more non-native interchain disulfide bond(s) that improves the stability of C1, C2 and/or the interface between C1 and C2.

4. The polypeptide complex or antigen-binding portion thereof of any of claims 1-3, wherein VH1 comprises the amino acid sequence of SEQ ID NO: 13 or an amino acid sequence with at least 85%, 90%, or 95% identity to SEQ ID NO: 13 and VL1 comprises the amino acid sequence of SEQ ID NO:14 or an amino acid sequence with at least 85%, 90%, or 95% identity to SEQ ID NO: 14; and/or
VH2 comprises the amino acid sequence of SEQ ID NO: 15 or an amino acid sequence with at least 85%, 90%, or 95% identity to SEQ ID NO: 15 and VL2 comprises the amino acid sequence of SEQ ID NO:16 or an amino acid sequence with at least 85%, 90%, or 95% identity to SEQ ID NO: 16.

5. The polypeptide complex or antigen-binding portion thereof of claim 3 or 4, wherein C1 comprises the amino acid sequence of SEQ ID NO: 17 or an amino acid sequence with at least 85%, 90%, or 95% identity to SEQ ID NO: 17, and C2 comprises the amino acid sequence of SEQ ID NO: 18 or an amino acid sequence with at least 85%, 90%, or 95% identity to SEQ ID NO: 18.

6. The polypeptide complex or antigen-binding portion thereof of any of the preceding claims, wherein the polypeptide complex comprises a Fc region, such as a human IgG Fc region.

7. The polypeptide complex or antigen-binding portion thereof of claim 6, wherein the Fc region is a human IgG1, IgG2, IgG3 or IgG4 Fc region.

8. The polypeptide complex or antigen-binding portion thereof of claim 6 or 7, wherein the Fc region is a native Fc region or an engineered Fc region, such as a Fc region comprising one or more substitutions selected from “knob into hole” substitutions, S228P, M252Y/S254T/T256E and combinations thereof.

9. The polypeptide complex or antigen-binding portion thereof of any of the preceding claims, comprising one, two or more CD47-binding moieties, as well as one, two or more HER2-binding moieties.

10. The polypeptide complex or antigen-binding portion thereof of claim 9, comprising two heavy chains and two light chains, wherein from the N-terminus to the C-terminus:
the first heavy chain comprises domains operably linked as in VH1-C1-hinge-Fe,
the second heavy chain comprises domains operably linked as in VH2-CH1-hinge-Fe,
the first light chain comprises domains operably linked as in VL1-C2, and
the second light chain comprises domains operably linked as in VL2-CL;
or
the first heavy chain comprises domains operably linked as in VH1-CH1-hinge-Fc,
the second heavy chain comprises domains operably linked as in VH2-C1-hinge-Fe,
the first light chain comprises domains operably linked as in VL1-CL, and
the second light chain comprises domains operably linked as in VL2-C2.

11. The polypeptide complex or antigen-binding portion thereof of claim 9, comprising two heavy chains and four light chains, wherein from the N-terminus to the C-terminus:
the first and second heavy chains each comprises domains operably linked as in VH1-C1-VH2-CH1-hinge-Fc, VH2-CH1-VH1-C1-hinge-Fe, VH1-C1-hinge-Fc-VH2-CH1, or VH2-CH1-hinge-Fc-VH1-C1,
the first and second light chains each comprises domains operably linked as in VL1-C2, and
the third and fourth light chains each comprises domains operably linked as in VL2-CL; or
the first and second heavy chains each comprises domains operably linked as in VH1-CH1-VH2-C1-hinge-Fe, VH2-C1-VH1-CH1-hinge-Fc, VH1-CH1-hinge-Fc-VH2-C1, or VH2-C1-hinge-Fc-VH1-CH1,
the first and second light chains each comprises domains operably linked as in VL1-CL, and
the third and fourth light chains each comprises domains operably linked as in VL2-C2.

12. The polypeptide complex or antigen-binding portion thereof of claim 9, comprising two heavy chains and three light chains, wherein from the N-terminus to the C-terminus:
the first heavy chain comprises domains operably linked as in VH1-C1-VH2-CH1-hinge-Fc or VH2-CH1-hinge-Fc-VH1-C1,
the second heavy chain comprises domains operably linked as in VH2-CH1-hinge-Fe,
the first light chain comprises domains operably linked as in VL1-C2, and
the second and third light chains each comprises domains operably linked as in VL2-CL; or
the first heavy chain comprises domains operably linked as in VH2-CH1-VH1-C1-hinge-Fc or VH1-C1-hinge-Fc-VH2-CH1,
the second heavy chain comprises domains operably linked as in VH1-C1-hinge-Fe,
the first and second light chains each comprises domains operably linked as in VL1-C2, and
the third light chain comprises domains operably linked as in VL2-CL;
or
the first heavy chain comprises domains operably linked as in VH1-CH1-VH2-C1-hinge-Fc or VH2-C1-hinge-Fc-VH1-CH1,
the second heavy chain comprises domains operably linked as in VH2-C1-hinge-Fe,
the first light chain comprises domains operably linked as in VL1-CL, and
the second and third light chains each comprises domains operably linked as in VL2-C2;
or
the first heavy chain comprises domains operably linked as in VH2-C1-VH1-CH1-hinge-Fc or VH1-CH1-hinge-Fc-VH2-C1,
the second heavy chain comprises domains operably linked as in VH1-CH1-hinge-Fc,
the first and second light chains each comprises domains operably linked as in VL1-CL, and
the third light chain comprises domains operably linked as in VL2-C2.

13. The polypeptide complex or antigen-binding portion thereof of any of claims 9-12, wherein the C1 and C2 domains are exchangeable.

14. The polypeptide complex or antigen-binding portion thereof of any of claims 3-13, wherein operably linked is via a direct linkage or via a peptide linker comprising 1-30 amino acids in length.

15. The polypeptide complex or antigen-binding portion thereof of claim 14, wherein the peptide linker is a GS linker, for example (GS)n, (GGS)n, (GGGS)n, (GGGGS)n, (GGSG)n, (GGGSS)n, wherein n is an integer of 1-9.

16. The polypeptide complex or antigen-binding portion thereof of claim 10, comprising: a first and a second heavy chain that comprise SEQ ID NOs: 19 and 20, respectively, and a first and a second light chain that comprise SEQ ID NOs: 21 and 22, respectively.

17. The polypeptide complex or antigen-binding portion thereof of any of the preceding claims, wherein the polypeptide complex is a humanized antibody or fully human antibody.

18. An isolated nucleic acid molecule, comprising a nucleic acid sequence encoding the polypeptide complex or antigen-binding portion thereof of any of claims 1-17.

19. A vector comprising the nucleic acid molecule of claim 18.

20. A host cell comprising the nucleic acid molecule of claim 18, or the vector of claim 19.

21. A pharmaceutical composition comprising the polypeptide complex or antigen-binding portion thereof of any of claims 1-17 and a pharmaceutically acceptable carrier.

22. A method for producing the polypeptide complex or antigen-binding portion thereof of any of claims 1-17, comprising the steps of:
culturing a host cell comprising a nucleic acid sequence encoding the polypeptide complex or antigen-binding portion thereof under suitable conditions; and
isolating the polypeptide complex from the host cell.

23. A method for modulating a HER2 and/or CD47 related immune response in a subject, comprising administering to the subject the polypeptide complex or antigen-binding portion thereof of any of claims 1-17 or the pharmaceutical composition of claim 21 to the subject.

24. A method for inhibiting growth of tumor cells in a subject, comprising administering an effective amount of the polypeptide complex or antigen-binding portion thereof of any of claims 1-17 or the pharmaceutical composition of claim 21 to the subject.

25. A method for diagnosing, preventing or treating cancer in a subject, comprising administering an effective amount of the polypeptide complex or antigen-binding portion thereof of any of claims 1-17 or the pharmaceutical composition of claim 21 to the subject.

26. The method of claim 25, wherein the cancer is an HER2 and/or CD47 positive cancer.

27. The method of claim 25 or 26, wherein the cancer is selected from colon cancer, colorectal cancer, breast cancer, lung cancer, cervical cancer, renal cancer, glioblastoma, ovarian cancer, pancreatic cancer, prostate cancer, esophageal cancer, gastric cancer, melanoma, liver cancer, head and neck cancer, skin cancer, bladder cancer, brain cancer, bronchial cancer, cancer of the bile duct, endometrial cancer, glioma, cancer of unknown primary origin, sarcoma, medulloblastoma, neuroblastoma, retinoblastoma, testicular cancer, thymoma, thyroid cancer, urachal cancer, uterine cancer, vaginal cancer, astrocytoma, basal cell carcinoma, and combinations thereof.

28. The method of claim 27, wherein the cancer is breast cancer or gastric cancer.

29. The method of any of claims 24-28, wherein the polypeptide complex or antigen-binding portion thereof of any of claims 1-17 is administered in combination with a chemotherapeutic agent, radiation and/or other agents for use in cancer immunotherapy.

30. The polypeptide complex or antigen-binding portion thereof of any of claims 1-17 for use in
i) modulating a HER2 and/or CD47 related immune response;
ii) inducing macrophage-mediated phagocytosis of tumor cells;
iii) inducing natural killer cell-mediated cytotoxicity of tumor cells; and/or
iv) inhibiting growth of tumor cells.

31. The polypeptide complex or antigen-binding portion thereof of any of claims 1-17 for use in diagnosing, treating or preventing cancer.

32. Use of the polypeptide complex or antigen-binding portion thereof of any of claims 1-17 in the manufacture of a medicament for
i) modulating a HER2 and/or CD47 related immune response;
ii) inducing macrophage-mediated phagocytosis of tumor cells;
iii) inducing natural killer cell-mediated cytotoxicity of tumor cells; and/or
iv) inhibiting growth of tumor cells.

33. Use of the polypeptide complex or antigen-binding portion thereof of any of claims 1-17 in the manufacture of a medicament for treating or preventing a cancer associated with CD47 and/or HER2.

34. A kit, wherein the kit comprises a container comprising the polypeptide complex or antigen-binding portion thereof of any of claims 1-17.
